(19) United States
(12) Patent Application Publication
(10) Pub. No.: US 2026/0273060 A1
(43) Pub. Date: Sep. 17, 2026

(54) iPSC-DERIVED NK CELL FOR LYMPHOMA TREATMENT

(71) Applicant: FATE THERAPEUTICS, INC., San Diego, CA (US)

(72) Inventors: Bahram VALAMEHR, San Diego, CA (US); ALAN WILLIAMS, San Diego, CA (US); JODE GOODRIDGE, San Diego, CA (US)

(21) Appl. No.: 19/473,115

(22) PCT Filed: Apr. 5, 2024
(86) PCT No.: PCT/US2024/023462

§ 371 Date: Oct. 6, 2025

Related U.S. Application Data
(60) Provisional application No. 63/495,068, filed on Apr. 7, 2023.

(51) Int. Cl. A61K 40/42; A61K 31/4184; A61K 31/675; A61K 31/7076; A61K 39/395; A61K 40/15; A61K 40/31; A61P 35/00

(52) U.S. Cl. CPC A61K 40/4211; A61K 31/4184; A61K 31/675; A61K 31/7076; A61K 39/3955; A61K 40/15; A61K 40/31; A61K 40/4217; A61P 35/00

(57) ABSTRACT

Provided are methods and compositions for use in cancer immunotherapies. In various embodiments, the compositions include functionally enhanced derivative effector cells obtained from directed differentiation of genomically engineered iPSCs. In various embodiments, the derivative cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATION

The application claims priority to U.S. Provisional Application Ser. No. 63/495,068, filed Apr. 7, 2023, the disclosure of which is hereby incorporated by reference in its entirety.

### INCORPORATED BY REFERENCE OF SEQUENCE LISTING

The Sequence Listing titled 184143-651601_SL.xml, which was created on Apr. 2, 2024 and is 19,392 bytes in size, is hereby incorporated by reference in its entirety.

### FIELD OF THE INVENTION

The present disclosure is broadly concerned with the field of off-the-shelf immunocellular products. More particularly, the present disclosure is concerned with strategies for developing multifunctional effector cells capable of delivering therapeutically relevant properties in vivo. The cell products developed under the present disclosure address critical limitations of patient-sourced cell therapies.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit. There is also the need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, significant opportunities remain to harness the full potential of T and NK cells, or other lymphocytes in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, (e.g., efficacy in a tumor microenvironment and related immune suppression, recruiting, trafficking and infiltration).

It is an object of embodiments of the present invention to provide methods and compositions for adoptive cell therapy, wherein the adoptive cell therapy includes administering an adoptive cell therapy product generated from derivative non-pluripotent cells differentiated from a single cell derived iPSC (induced pluripotent stem cell) clonal line, which iPSC line comprises one or several genetic modifications in its genome. Said one or several genetic modifications include, in some embodiments, one or more of DNA insertion, deletion, and substitution, and which modifications are retained and remain functional in subsequently derived cells after differentiation, expansion, passaging and/or transplantation.

One aspect of the present invention provides a method of treating a subject having lymphoma, and the method comprises administering to the subject at least a first cycle of an adoptive cell therapy product, with the first cycle comprising one or more doses of the adoptive cell therapy product administered in a first effective amount at a preselected frequency, and with an option of administering one or more additional cycles with one or more doses in a second effective amount, during a course of treatment over a period of time, wherein the first and the second effective amounts are the same or different; wherein the adoptive cell therapy product comprises an engineered natural killer (NK) lineage cell comprising CD19-CAR (chimeric antigen receptor) expression, 41BB-ADR expression, exogenous CD16 expression, IL15RF expression, and CD38 knockout; and wherein the lymphoma expresses CD19.

In one embodiment of said method, the lymphoma is (i) relapsed and/or refractory B-cell lymphoma (R/R BCL); (ii) expresses CD20; and/or (iii) is follicular lymphoma (FL), non-Hodgkin lymphoma (NHL), transformed indolent NHL, diffuse large B cell lymphoma (DLBCL), high-grade BCL (HGBCL), or primary mediastinal BCL. In some embodiments of the method, the subject has received at least one prior line of treatment comprising: (i) an anti-CD20 monoclonal antibody therapy; (ii) a CAR T-cell therapy; and/or (iii) one of rituximab, obinutuzumab, chlorambucil, fludarabine, bendamustine, Hyper-CVAD, R-CHOP, or RDHAP, ibrutinib, acalabrutinib, zanubrutinib, pirtobrutinib, bortezomib venetoclax, lenalidomide, or combinations thereof. In some other embodiments, the subject had no prior line of treatment. In some embodiments, the subject had no prior CAR T-cell treatment.

In some embodiments of the method, the course of treatment further comprises administering to the subject an effective amount of an anti-CD20 antibody. In some other embodiments, the course of treatment comprising an anti-CD20 antibody further comprises a conditioning chemotherapy. In some embodiments, the anti-CD20 antibody comprises rituximab or a biosimilar thereof. In some embodiments, the conditioning chemotherapy comprises: (i) cyclophosphamide (CY); (ii) fludarabine (FLU); or (iii) bendamustine. In some embodiments, the conditioning chemotherapy comprises: (i) cyclophosphamide (CY) and (ii) fludarabine (FLU). In some embodiments of the method, the course of treatment comprising the anti-CD20 antibody does not comprise a conditioning chemotherapy.

In some embodiments, the anti-CD20 antibody is administered at a starting time prior to each of the first cycle and the one or more additional cycles of administering the adoptive cell therapy product. In some embodiments of the method, the conditioning chemotherapy is administered prior to day 1 of each cycle of administering the adoptive cell therapy product. On day 1 of each cycle, the first dose of the adoptive cell therapy product is administered. Some embodiments of the method comprise administering the first and one or more additional cycles of the adoptive cell therapy over about 29 days with one dose each on day 1, 4, and 8.

In some embodiments of the method, the anti-CD20 antibody is rituximab or a biosimilar thereof, and is administered at a single dose of about 300 mg/m² to about 450 mg/m² to the subject about 2-6 days prior to day 1 of administering the adoptive cell therapy product in each cycle.

In some embodiments of the method, the conditioning chemotherapy comprises cyclophosphamide and is administered at a daily dose of about 250 mg/m² to about 600 mg/m² and fludarabine at a daily dose of about 20 mg/m² to about 40 mg/m² for 3 consecutive days starting from about 4-6 days prior to day 1 of administering the adoptive cell therapy product in each cycle.

In some other embodiments of the method, the conditioning chemotherapy comprises bendamustine and is administered at a daily dose of about 30 mg/m² to about 100 mg/m² for 2 consecutive days starting from about 4-6 days prior to day 1 of administering the adoptive cell therapy product in each cycle.

In some embodiments of the method, the engineered NK lineage cell is derived from an engineered induced pluripotent stem cell (iPSC) comprising a polynucleotide encoding a CD19-CAR, a polynucleotide encoding a 41BB-CAR, a polynucleotide encoding an exogenous CD16, a polynucleotide encoding IL15RF, and CD38 knockout.

In some embodiments of the method, the first and second effective amounts of the adoptive cell therapy product in each dose is about 1×10⁸ cells to about 3×10⁹ cells, and is optionally escalated. In some embodiments, the first and second effective amounts of the adoptive cell therapy product in each dose is about 1×10⁸ cells, about 3×10⁸ cells, about 9×10⁸ cells, about 2×10⁹ cells or about 3×10⁹ cells. In some embodiments of the method, if the effective amount in each dose results in a dose limiting toxicity (DLT) rate of 25% with an equivalence interval of 20%-30%, then the effective amount increases by 3 times or less to a higher dose, for example, to about 4×10⁹ cells or less, to about 6×10⁹ cells or less, to about 8×10⁹ cells or less, to about 9×10⁹ cells or less, or to about 10×10⁹ cells or less.

In one embodiment of the method, the administering of the adoptive cell therapy product is (i) via intravenous infusion, and/or (ii) at a site of an outpatient setting; and/or wherein each dose of the adoptive cell therapy product is cryopreserved, and then thawed prior to administering; and wherein the adoptive cell therapy product is FT522.

Another aspect of the application provides use of said method for slowing progression of and/or treating BCL in a subject. In some embodiments, the subject has R/R BCL. In some embodiments, the subject having BCL had no prior treatments. In some other embodiments, the subject having BCL had a CAR T-cell therapy for BCL. In some embodiments of using the above method for slowing progression or treating BCL, the method further comprises detecting and comparing one or more of the following at different given time points following administration of a first dose of the adoptive cell therapy: (a) the presence of the engineered immune cell in a tumor of the subject; (b) protein markers of disease in serum of the subject; (c) cytokines in a peripheral blood sample from the subject; (d) circulating tumor DNA in a peripheral blood sample from the subject; and (e) lesion size and/or number, wherein any of (a)-(e) is used to assess tumor burden, tumor immunobiology, and/or tumor therapy response, thereby determining efficacy of the multi-dose targeted adoptive cell therapy.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIG. 1 shows an exemplary treatment schema to evaluate FT522 as an allogeneic cell therapy with one or more treatment cycles in combination with a secondary antigen targeting monoclonal antibody, and an optional conditioning chemotherapy. Abbreviations in FIG. 1 are as follows: CY (cyclophosphamide), DL (dose level), DLT (dose-limiting toxicity), FLU (fludarabine), LTFU (long-term follow-up), PTFU (post-treatment follow-up), y (year).

FIG. 2 shows FT522 dose-escalation and dose expansion schema. Additional dose levels >DL3 (≤3× the prior cleared dose level) may be explored. Conditioning^{a}: Standard conditioning chemotherapy with CY/FLU (Regimen A only); bendamustine may be used instead of CY/FLU with approval from the medical monitor. DL1^{b}: Regimen B may open at the highest cleared dose in Regimen A. Abbreviations in FIG. 2 are as follows: CAR T (Chimeric antigen receptor T-cell therapy), CY (Cyclophosphamide), BCL (B-cell lymphoma), DL (dose level), DLBCL (diffuse large B-cell lymphoma), FL (follicular lymphoma), FLU (fludarabine), MAD (maximum assessed dose), MTD (maximum tolerated dose), R (randomized), R/R (relapsed/refractory).

### DETAILED DESCRIPTION OF THE INVENTION

Genomic modification of iPSCs (induced pluripotent stem cells) can include one or more of polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. In various embodiments, the present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cells), T cell progenitor cells, NK cell progenitor cells, T cells, NKT cells, NK cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or #1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition at a functionally inert, low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refer to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, refers to stem cells that are produced in vitro from differentiated adult, neonatal or fetal cells that have been induced or changed, i.e., reprogrammed into cells capable of differentiating into tissues of all three germ or dermal layers: mesoderm, endoderm, and ectoderm. In some embodiments, the reprogramming process uses reprogramming factors and/or small molecule chemical driven methods. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta (i.e., are not totipotent).

As used herein, the term “multipotent stem cell” refers to a cell that has the developmental potential to differentiate into cells of one or more germ layers (i.e., ectoderm, mesoderm and endoderm), but not all three. Thus, a multipotent cell can also be termed a “partially differentiated cell.” Multipotent cells are known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to the state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “naïve” or “ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The terms “hematopoietic stem and progenitor cells,” “hematopoietic stem cells,” “hematopoietic progenitor cells,” or “hematopoietic precursor cells” refer to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakaryocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be a CD3⁺ cell. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The term “T cell” can also refer to a genetically engineered T cell, such as a T cell modified to express a T cell receptor (TCR) or a chimeric antigen receptor (CAR). A T cell or T cell-like effector cell can also be differentiated from a stem cell or progenitor cell (“a derived T cell” or “a derived T cell like effector cell”, or collectively, “a derivative T lineage cell”). A derived T cell like effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”. In some embodiments, the derivative T lineage cell is an iPSC-derived T cell obtained by differentiating an iPSC, which cells are also referred to herein as “iT” cells.

“CD4⁺ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” molecules are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56+NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell-like effector cell may be differentiated from a stem cell or progenitor cell (“a derived NK cell” or “a derived NK cell like effector cell”, or collectively, “a derivative NK lineage cell”). A derivative NK cell like effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”. In some embodiments, the derivative NK lineage cell is an iPSC-derived NK cell obtained by differentiating an iPSC, which cells are also referred to herein as “INK” cells.

As used herein, the term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified with the expression of at least one or more of the following markers, TCR Va24-Ja18, Vb11, CD1d, CD3, CD4, CD8, aGalCer, CD161 and CD56.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an “isolated cell” is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained by separating the desired cells, or populations thereof, from other substances or cells in the environment, or by removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as “encoding” the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. Thus, the term “vector” comprises the construct to be delivered. A vector can be a linear or a circular molecule. A vector can be integrating or non-integrating. The major types of vectors include, but are not limited to, plasmids, episomal vectors, viral vectors, cosmids, and artificial chromosomes. Viral vectors include, but are not limited to, adenovirus vectors, adeno-associated virus vectors, retrovirus vectors, lentivirus vectors, Sendai virus vectors, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or is non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK lineage cells or derivative T lineage cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSCs using genomic editing. In the aspect of a source cell obtained from a specifically selected donor, disease or treatment context, the genetic imprint contributing to preferential therapeutic attributes may include any context-specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements (e.g., selected HLA-matched donor cells exhibiting a haplotype with increased population). As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; and/or resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. For example, synthetic effector cells, or synthetic NK cells or synthetic T cells, as used throughout this application are cells differentiated from a genomically modified iPSC, as compared to their primary counterpart obtained from natural/native sources such as peripheral blood, umbilical cord blood, or other donor tissues. In some embodiments, a synthetic cell possesses one or more non-native cell functions when compared to its closest counterpart primary cell.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; and/or resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, a neutrophil), and a tumor cell; and activating the immune cell. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, an NK cell, an NKT cell, a B cell, a macrophage, or a neutrophil. In some embodiments, the surface triggering receptor facilitates bi- or multi-specific antibody engagement between the effector cells and a specific target cell (e.g., a tumor cell) independent of the effector cells' natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some cases, and to kill two or more types of tumors in some other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch protein could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. Pharmaceutically active proteins include an entire protein or peptide or pharmaceutically active fragments thereof. The term also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, cellular signal transduction. “Signal transduction” refers to the transmission of a molecular signal in the form of chemical modification by recruitment of protein complexes along a pathway that ultimately triggers a biochemical event in the cell. Examples of signal transduction pathways are known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes but is not limited to (i) antigen specificity as it relates to a unique chimeric antigen receptor (CAR) or T cell receptor (TCR), (ii) engager specificity as it relates to monoclonal antibodies or bispecific engagers, (iii) targeting of transformed cells, (iv) targeting of cancer stem cells, and (v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that relates to the transfusion of autologous or allogeneic lymphocytes, whether the immune cells are isolated from a human donor, or effector cells obtained from in vitro differentiation of a pluripotent cell; whether they are genetically modified or not; or whether they are primary donor cells or cells that have been passaged, expanded, or immortalized, ex vivo, after isolation from a donor.

As used herein, “lymphodepletion” and “lympho-conditioning” are used interchangeably to refer to the destruction of lymphocytes and T cells, typically prior to immunotherapy. The purpose of lympho-conditioning prior to the administration of an adoptive cell therapy is to promote homeostatic proliferation of effector cells as well as to eliminate regulatory immune cells and other competing elements of the immune system that compete for homeostatic cytokines. Thus, lympho-conditioning is typically accomplished by administering one or more chemotherapeutic agents to the subject prior to a first dose of the adoptive cell therapy. In various embodiments, lympho-conditioning precedes the first dose of the adoptive cell therapy by a few hours to a few days. Exemplary chemotherapeutic agents useful for lympho-conditioning include, but are not limited to, cyclophosphamide (CY), fludarabine (FLU), and those described below. However, a sufficient lymphodepletion through anti-CD38 mAb could provide an alternative conditioning process (e.g., for use in an iNK cell therapy in accordance with various embodiments herein), without or with minimal need of a CY/FLU-based lympho-conditioning procedure, as further described herein.

As used herein, the term “outpatient” refers to a patient who is not hospitalized overnight, but who visits a hospital, clinic, or associated facility for diagnosis and/or treatment. Thus, an “outpatient setting,” as compared to an “inpatient setting” refers to an environment for providing ambulatory care or outpatient care to a patient where hospitalization for one or more days/nights is not required while receiving treatment and/or diagnosis, thereby reducing overall discomfort to the patient receiving treatment and/or diagnosis, while reducing overall cost for such treatment and/or diagnosis with relative ease in management and coordination. Additionally, an outpatient setting is more readily accessible to a larger population of patients and increases patient availability and patient compliance with a treatment protocol during a trial or course of treatment.

As used herein, “induction therapy,” also called “first-line therapy,” “primary therapy,” or “primary treatment,” refers to a first treatment given to a patient for a particular disease. It is often part of a standard set of treatments, such as surgery followed by chemotherapy and radiation. Thus, an “induction attempt” or “attempt of induction therapy” refers to an initial attempt at treating a particular disease using known and/or conventional therapeutic approaches for the particular disease.

A “therapeutically sufficient amount,” as used herein, includes within its meaning a non-toxic but sufficient and/or effective amount of a particular therapeutic agent and/or pharmaceutical composition to which it is referring to provide a desired therapeutic effect. The exact amount required will vary from subject to subject, depending on factors such as the patient's general health, the patient's age and the stage and severity of the condition being treated. In particular embodiments, a therapeutically sufficient amount is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (e.g., ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to the differentiation cues within the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates, forming larger aggregates, which can be mechanically or enzymatically dissociated into smaller aggregates to maintain cell proliferation within the culture and increase numbers of cells. As distinct from EB culture, cells cultured within aggregates in maintenance culture media maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells (e.g., EB formation). Monolayer differentiation, among other advantages disclosed herein, avoids the need for EB formation to initiate differentiation. Because monolayer culturing does not mimic embryo development such as is the case with EB formation, differentiation towards specific lineages is deemed to be minimal as compared to all three germ layer differentiation in EB formation.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be enzymatically or mechanically dissociated from each other, such as by dissociation into a suspension of clusters, single cells or a mixture of single cells and clusters. In yet another alternative embodiment, adherent cells can be dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level-successful removal, addition, or alteration of a cell function/characteristic via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduces a signaling response is “antagonistic” or “an antagonist.”

The term “antibody” encompasses antibodies and antibody fragments that contain at least one binding site that specifically binds to a particular target of interest, wherein the target may be an antigen, or a receptor that is capable of interacting with certain antibodies. The term “antibody” includes, but is not limited to, an immunoglobulin molecule or an antigen-binding or receptor-binding portion thereof. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term antibody also includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, single variable new antigen receptor (VNAR), shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated “FcR”, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signaling properties of each receptor. Fc-gamma receptors (FcγR) includes several members, FcγRI (CD64), FcγRIIA (CD32), FcγRIIB (CD32), FcγRIIIA (CD16a), FcγRIIIB (CD16b), which differ in their antibody affinities due to their different molecular structures.

“Chimeric Fc Receptor,” abbreviated as “CFcR,” is a term used to describe engineered Fc receptors having their native transmembrane and/or intracellular signaling domains modified, or replaced with non-native transmembrane and/or intracellular signaling domains. In some embodiments of the chimeric Fc receptor, in addition to having one of, or both, transmembrane and signaling domains being non-native, one or more stimulatory domains can be introduced to the intracellular portion of the engineered Fc receptor to enhance cell activation, expansion and function upon triggering of the receptor. Unlike chimeric antigen receptor (CAR) which contains antigen binding domain to target antigen, the chimeric Fc receptor binds to an Fc fragment, or the Fc region of an antibody, or the Fc region comprised in an engager or a binding molecule and activating the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fcγ receptor, by replacing its transmembrane domain and/or intracellular domain. To further improve the binding affinity of the CD16 based CFcR, the extracellular domain of CD64 or the high-affinity variants of CD16 (F176V, for example) can be incorporated. In some embodiments of the CFcR where high affinity CD16 extracellular domain is involved, the proteolytic cleavage site comprising a serine at position 197 is eliminated or is replaced such at the extracellular domain of the receptor is non-cleavable, i.e., not subject to shedding, thereby obtaining a hnCD16 based CFcR.

CD16, a FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in WO2015/148926 and U.S. Pat. No. 10,464,989, the complete disclosures of which are incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

As used herein, “FT522” refers to a multiplexed engineered NK cell therapy generated from a clonal master engineered iPSC line, and is engineered with four modalities for enhanced innate immunity: (1) a high-affinity non-cleavable CD16 (hnCD16) Fc receptor; (2) IL15/IL15 receptor fusion (IL15RF); (3) anti-CD19 CAR; (4) 41BB-ADR and (5) CD38 knockout.

I. Cells and Compositions Useful for Adoptive Cell Therapies with Enhanced Properties

Provided herein is a strategy to systematically engineer the regulatory circuitry of a clonal iPSC without impacting the differentiation potency of the iPSC and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modulated activities. Unanticipated failures during directed cell differentiation from iPSCs have been attributed to aspects including, but not limited to, development stage specific gene expression or lack thereof, requirements for HLA complex presentation, protein shedding of introduced surface expressing modalities, and need for reconfiguration of differentiation protocols enabling phenotypic and/or functional changes in the cell. The present application has shown that the one or more selected genomic modifications as provided herein does not negatively impact iPSC differentiation potency, and the functional effector cells derived from the engineered iPSC have enhanced and/or acquired therapeutic properties attributable to the individual or combined genomic modifications retained in the effector cells following the iPSC differentiation.

### 1. ADR Armed CAR-19 NK Cells

Unwanted activation of T- and NK-cells often promotes allo-immune reactions leading to development of graft-versus-host disease (GvHD). Although some steps may be taken to reduce the reactivity of allogeneic cells in the recipient individual, such cells would still be targeted by the immune system of the recipient (primarily T- and NK-cells), which would recognize them as foreign leading to rejection and limiting therapeutic benefit. On the other hand, modulating a host immune system to reduce allo-immune reactions, for example, by lympho-conditioning using chemotherapy such as Cy/Flu (cyclophosphamide/fludarabine) often leads to associated hematologic toxicities, including increased susceptibility to severe infections, due to indiscrimitive lymphodepletion and a severely compromised immune system as a result. To control pathogenic conditions due to unwanted activation of the immune system, in various embodiments, the present application provides a solid tumor targeting backbone comprising an allo-immune defense receptor (ADR), among other components. Another aspect of the application provides immune cells, iPSCs, and iPSC-derived effector cells that are genetically engineered to comprise, among other editing as contemplated and described herein, a 4-1BB or CD38 specific allo-immune defense receptor (ADR) for effector cell potentiation as well as selective depletion of alloreactive host NK cells and T cells with upregulated 4-1BB and/or CD38 expression, the latter of which include pathogenic T cells, and regulatory T cells, while sparing resting cells in the recipient.

In some embodiments of the ADR that is specific to 4-1BB (CD137, also referred to as “41BB”), the ADR comprises an extracellular domain that targets 4-1BB upregulated on host T or NK cells when they are activated, and a signaling domain promoting effector cell activation. For example, the 41BB-ADR extracellular domain may comprise any suitable ligand for 4-1BB, including 4-1BBL, an antibody (or functional fragment thereof) that targets 4-1BB, a fusion of Fc with 4-1BBL, or functional derivatives or fragments thereof. In some embodiments, the 41BB-ADR extracellular domain comprises 4-1BBL, or a fragment thereof effective to bind 4-1BB. In some embodiments, the 41BB-ADR extracellular domain comprises an amino acid sequence with at least about 85%, about 90%, about 95%, about 96%, about 97%, about 98%, or about 99% sequence identity to SEQ ID NO: 1. In some embodiments, the 41BB-ADR extracellular domain comprises an amino acid sequence with at least about 90% sequence identity to SEQ ID NO: 1. In some embodiments, the 41BB-ADR extracellular domain comprises an amino acid sequence with at least about 95% sequence identity to SEQ ID NO: 1. In some embodiments, the 41BB-ADR extracellular domain comprises the amino acid sequence of SEQ ID NO: 1.

SEQ ID NO: 1
GLLDLRQGMFAQLVAQNVLLIDGPLSWYSDPGLAGVSLTGGLSYKEDTKE
LVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGA
TVLGLFRVTPEIPAGLPSPRSE

In particular embodiments, the ADR comprises CD3ζ, represented by an amino acid sequence of at least about 85%, about 90%, about 95%, about 96%, about 97%, about 98%, or about 99% sequence identity to SEQ ID NO: 2 or a functional fragment thereof, or comprises a CD3ζ derivative (for example, CD3ζ1XX, represented by an amino acid sequence of at least about 85%, about 90%, about 95%, about 96%, about 97%, about 98%, or about 99% sequence identity to SEQ ID NO: 3 or a functional fragment thereof). In some embodiments, the CD3ζ comprises an amino acid sequence of at least about 90% sequence identity to SEQ ID NO: 2. In some embodiments, the CD3ζ comprises an amino acid sequence of at least about 95% sequence identity to SEQ ID NO: 2. In some embodiments, the CD3ζ comprises the amino acid sequence of SEQ ID NO: 2. In some embodiments, the CD3ζ derivative comprises an amino acid sequence of at least about 90% sequence identity to SEQ ID NO: 3. In some embodiments, the CD3ζ derivative comprises an amino acid sequence of at least about 95% sequence identity to SEQ ID NO: 3. In some embodiments, the CD3ζ derivative comprises the amino acid sequence of SEQ ID NO: 3. CD32 mediates downstream ITAM-derived signaling during effector T or NK cell activation.

SEQ ID NO: 2
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
(CD3ζ)
SEQ ID NO: 3
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR
(CD3ζ1XX-containing 2 mutations in ITAM1)

In one embodiment of the 4-1BB specific ADR, the 41BB-ADR is represented by an amino acid sequence of at least about 85%, about 90%, about 95%, about 96%, about 97%, about 98%, or about 99% sequence identity to SEQ ID NO: 4 or SEQ ID NOs: 5-7. In some embodiments, the 41BB-ADR comprises an amino acid sequence of at least about 90% identity to SEQ ID NO: 4 or SEQ ID NOs: 5-7. In some embodiments, the 41BB-ADR comprises an amino acid sequence of at least about 95% identity to SEQ ID NO: 4 or SEQ ID NOs: 5-7. In some embodiments, the 41BB-ADR comprises the amino acid sequence of SEQ ID NO: 4. In some embodiments, the 41BB-ADR comprises the amino acid sequence of SEQ ID NO: 5. In some embodiments, the 41BB-ADR comprises the amino acid sequence of SEQ ID NO: 6. In some embodiments, the 41BB-ADR comprises the amino acid sequence of SEQ ID NO: 7.

SEQ ID NO: 4
MEFGLSWLFLVAILKGVQCGLLDLRQGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTVDLPPASSEARNSAFGFQGRL
LHLSAGQRLGVHLHTEARARHAWQLTQGATVLGLFRVTPEIPAGLPSPRSEESKYGPPCPPCPGQPREPQ
VYTLPPSRDELTKNQVSLTCLVKGFYPSDIAVEWESNGQPENNYKTTPPVLDSDGSFFLYSKLTVDKSRW
QQGNVFSCSVMHEALHNAYTQKSLSLSPGKKDPKFWVLVVVGGVLACYSLLVTVAFIIFWVRSRVKFSRS
ADAPAYQQGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
Signal peptide-41BBL-spacer-CD28 (TM)-CD3z (the signal peptide, spacer
and TM/transmembrane domain may vary)
SEQ ID NO: 5
MEFGLSWLFLVAILKGVQCGLLDLRQGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTVDLPPASSEARNSAFGFQGRL
LHLSAGQRLGVHLHTEARARHAWQLTQGATVLGLFRVTPEIPAGLPSPRSEESKYGPPCPPCPGQPREPQ
VYTLPPSRDELTKNQVSLTCLVKGFYPSDIAVEWESNGQPENNYKTTPPVLDSDGSFFLYSKLTVDKSRW
QQGNVFSCSVMHEALHNAYTQKSLSLSPGKKDPKFWVLVVVGGVLACYSLLVTVAFIIFWVRSKRSRLLH
SDYMNMTPRRPGPTRKHYQPYAPPRDFAAYRSRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKDTYDALHMQA
LPPR
Signal peptide-41BBL-spacer-CD28 (TM)-CD28 (ICD)-CD3z (the signal
peptide, spacer and TM/transmembrane domain may vary)
SEQ ID NO: 6
MEFGLSWLFLVAILKGVQCGLLDLRQGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTVDLPPASSEARNSAFGFQGRL
LHLSAGQRLGVHLHTEARARHAWQLTQGATVLGLFRVTPEIPAGLPSPRSEESKYGPPCPPCPGQPREPQ
VYTLPPSRDELTKNQVSLTCLVKGFYPSDIAVEWESNGQPENNYKTTPPVLDSDGSFFLYSKLTVDKSRW
QQGNVFSCSVMHEALHNAYTQKSLSLSPGKKDPKFWVLVVVGGVLACYSLLVTVAFIIFWVRSRVKFSRS
ADAPAYQQGQNQLYNELNLGRREEYDVLDKRRGRDPEMGGKPRRKNPQEGLFNELQKDKMAEAFSEIGMK
GERRRGKGHDGLFQGLSTATKDTFDALHMQALPPR
Signal peptide-41BBL-spacer-CD28 (TM)-CD3z1xx (the signal peptide,
spacer and TM/transmembrane domain may vary)
SEQ ID NO: 7
MEFGLSWLFLVAILKGVQCGLLDLRQGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTVDLPPASSEARNSAFGFQGRL
LHLSAGQRLGVHLHTEARARHAWQLTQGATVLGLFRVTPEIPAGLPSPRSEESKYGPPCPPCPGQPREPQ
VYTLPPSRDELTKNQVSLTCLVKGFYPSDIAVEWESNGQPENNYKTTPPVLDSDGSFFLYSKLTVDKSRW
QQGNVFSCSVMHEALHNAYTQKSLSLSPGKKDPKFWVLVVVGGVLACYSLLVTVAFIIFWVRSKRSRLLH
SDYMNMTPRRPGPTRKHYQPYAPPRDFAAYRSRVKFSRSADAPAYOQGQNQLYNELNLGRREEYDVLDKR
RGRDPEMGGKPRRKNPQEGLFNELQKDKMAEAFSEIGMKGERRRGKGHDGLFQGLSTATKDTFDALHMQA
LPPR
Signal peptide-41BBL-spacer-CD28 (TM)-CD28 (ICD)-CD3z1xx (the signal
peptide, spacer and TM/transmembrane domain may vary)

### 2, CD38 Knockout

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include phagocytosis (ADCP) and/or complement-dependent cytotoxicity (CDC).

Other than being highly expressed on malignant cells, CD38 is also expressed on plasma cells, as well as on NK cells, and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the calcium dependent process of cell adhesion. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38 specific antibody, NK cell reduction in both bone marrow and peripheral blood was observed, although other immune cell types, such as T cells and B cells, were unaffected despite their CD38 expression (Casneuf et al., Blood Advances. 2017; 1 (23):2105-2114).

Without being limited by theories, the present application includes a strategy to leverage the full potential of CD38 targeted cancer treatment by overcoming CD38 specific antibody and/or CD38 antigen binding domain induced effector cell depletion or reduction through fratricide. In addition, since CD38 is upregulated on activated lymphocytes such as T or B cells, by suppressing and/or eliminating these activated lymphocytes using an anti-CD38 antibody such as daratumumab in the recipient of allogeneic effector cells, host allorejection against these effector cells would be reduced and/or prevented, thereby increasing effector cell survival and persistency. As such, a CD38 antagonist, such as an anti-CD38 antibody, a secreted CD38 specific engager or a CD38-CAR (chimeric antigen receptor) against activation of recipient T, Treg, NK, and/or B cells can be used as a replacement for lymphodepletion using chemotherapy such as Cy/Flu (cyclophosphamide/fludarabine) prior to adoptive cell transferring.

In addition, when targeting CD38⁺ T and pbNK cells using CD38⁻ effector cells in the presence of anti-CD38 antibodies or CD38 inhibitors, the depletion of CD38⁺ alloreactive cells increases the NAD⁺ (nicotinamide adenine dinucleotide, a substrate of CD38) availability and decreases NAD⁺ consumption related cell death, which, among other advantages, boosts effector cell responses in an immunosuppressive tumor microenvironment and supports cell rejuvenation in aging, degenerative or inflammatory diseases.

Embodiments provided herein, such as for CD38 knockout, are compatible with other components and processes contemplated herein. Some embodiments include generating an iPSC line having CD19 targeting specificity described herein and a CD38 knockout, a master cell bank comprising single cell sorted and expanded clonal iPSCs, and obtaining CD38 negative (CD38^{neg} or CD38^{-/-}) derivative effector cells comprising the CD19 targeting specificity described herein through directed differentiation of the engineered iPSC line. In some embodiments, the derivative effector cells are protected against fratricide and allorejection when CD38 targeted therapeutic moieties are employed with the effector cells among other advantages including improved metabolic fitness, increased resistance to oxidative stress and inducing a protein expression program in the effector cell that enhances cell activation and effector function. In addition, anti-CD38 monoclonal antibody therapy significantly depletes a patient's activated immune system without adversely affecting the patient's hematopoietic stem cell compartment. A CD38^{neg} derivative cell has the ability to resist CD38 antibody mediated depletion, and may be effectively administered in combination with an anti-CD38 antibody or CD38-CAR without the use of toxic conditioning agents and thus reduce and/or replace chemotherapy-based lymphodepletion.

In one embodiment as provided herein, the CD38 knockout in an iPSC line is a bi-allelic knockout. In some embodiments of the construct, the construct comprises a pair of CD38 targeting homology arms for position-selective insertion within the CD38 locus. In some embodiments, the preselected targeting site is within an exon of CD38. The CD38-KI/KO constructs provided herein allow the transgene(s) to express either under the CD38 endogenous promoter or under an exogenous promoter comprised in the construct. When two or more transgenes are to be inserted at a selected location in the CD38 locus, a linker sequence, for example, a 2A linker or IRES, is placed between any two transgenes. The 2A linker encodes a self-cleaving peptide derived from FMDV, ERAV, PTV-I, and TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. The exogenous promoter comprised in a CD38-KI/KO construct may be CAG, or other constitutive, inducible, temporal-, tissue-, or cell type-specific promoters including, but not limited to CMV, EF1α, PGK, and UBC.

### 3. CD16 Knock-In

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” “high affinity non-cleavable CD16,” or “hnCD16,” as used herein, refers to various CD16 variants. The wildtype CD16 has low affinity and is subject to ectodomain shedding, a proteolytic cleavage process that regulates the cells surface density of various cell surface molecules on leukocytes upon NK cell activation. F176V (also called F158V in some publications) is an exemplary CD16 polymorphic variant having high affinity; whereas an S197P variant is an example of a genetically engineered non-cleavable version of CD16. An engineered CD16 variant comprising both F176V and S197P has high affinity and is non-cleavable, which was described in greater detail in International Pub. No. WO2015/148926 and U.S. Pat. No. 10,464,989, the complete disclosures of which are incorporated herein by reference. In some embodiments, the hnCD16 comprises an amino acid sequence of at least about 90% identity to SEQ ID NO: 8. In some embodiments, the hnCD16 comprises an amino acid sequence of at least about 95% identity to SEQ ID NO: 8. In some embodiments, the hnCD16 comprises the amino acid sequence of SEQ ID NO: 8.

SEQ ID NO: 8
MWQLLLPTALLLLVSAGMRTEDLPKAVVFLEPQWYRVLEKDSVTL
KCQGAYSPEDNSTQWFHNESLISSQASSYFIDAATVDDSGEYRCQ
TNLSTLSDPVQLEVHIGWLLLQAPRWVFKEEDPIHLRCHSWKNTA
LHKVTYLQNGKGRKYFHHNSDFYIPKATLKDSGSYFCRGLVGSKN
VSSETVNITITQGLAVPTISSFFPPGYQVSFCLVMVLLFAVDTGL
YFSVKTNIRSSTRDWKDHKFKWRKDPQDK

In some embodiments, the primary-sourced or derived effector cells comprising the exogenous CD16 or variant thereof are NK lineage cells. In some embodiments, the exogenous CD16 or functional variants thereof comprised in iPSC or effector cells has high affinity in binding to a ligand that triggers downstream signaling upon such binding. Non-limiting examples of ligands binding to the exogenous CD16 or functional variants thereof include not only ADCC antibodies or fragments thereof, but also to bi-, tri-, or multi-specific engagers or binders that recognize the CD16 extracellular binding domains of the exogenous CD16. Examples of bi-, tri-, or multi-specific engagers or binders are further described below in this application. As such, at least one of the aspects of the present application provides a derivative effector cell or a cell population thereof, preloaded with one or more pre-selected ADCC antibodies through an exogenous CD16 expressed on the derivative effector cell, in an amount sufficient for therapeutic use in a treatment of a condition, a disease, or an infection as further detailed this application, wherein the exogenous CD16 comprises an extracellular binding domain of a CD16 having F176V and S197P.

Unlike the endogenous CD16 expressed by primary NK cells which gets cleaved from the cellular surface following NK cell activation, the various non-cleavable versions of CD16 in derivative NK avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in derived NK cells also enables in vitro loading of an ADCC antibody to the NK cell through hnCD16 before administering the cell to a subject in need of a cell therapy.

In some embodiments, the derived NK cells comprising CD19 tumor antigen targeting specificity as described herein, have additional tumor antigen targeting specificity via an exogenous CD16 or a variant thereof that mediates ADCC when in combination with an antibody. In some embodiments, the derived NK cells comprising CD19 tumor antigen targeting specificity described herein further comprise CD38 knockout. In some embodiments, the derived NK cells comprising CD19 tumor antigen targeting specificity described herein and CD38 knockout, are in combination with a CD38 antibody. In some embodiments, the CD38 antibody is daratumumab. In some embodiments, the derived NK cells comprising CD19 tumor antigen targeting specificity described herein and CD38 knockout, are in combination with one or more of an anti-EGFR antibody (e.g., cetuximab, amivantamab), an anti-HER2 antibody (e.g., trastuzumab or biosimilars, pertuzumab), an anti-PDL1 antibody (e.g., avelumab), or a bi-specific antibody targeting EGFR and MET (e.g., amivantamab).

### 4. Exogenously Introduced Cytokine Signaling Complex

By avoiding systemic high-dose administration of clinically relevant cytokines, the risk of dose-limiting toxicities due to such a practice is reduced while cytokine mediated cell autonomy being established. To achieve lymphocyte autonomy without the need to additionally administer soluble cytokines, a cytokine signaling complex comprising a partial or full peptide of one or more of IL2, IL4, IL6, IL7, IL9, IL10, IL11, IL12, IL15, IL18, IL21, and/or their respective receptors may be introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling (signaling complex) are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine/cytokine receptor is through an expression construct carried by a retrovirus, Sendai virus, an adenovirus, an episome, mini-circle, or RNAs, including mRNA.

For example, in embodiments where the signaling complex is for IL15, the transmembrane (TM) domain can be native to the IL15 receptor or may be modified or replaced with transmembrane domain of any other membrane bound proteins. In various embodiments, the cytokine signaling complex comprises an IL15 receptor fusion (IL15RF) comprising a full or partial length of IL15 and a full or partial length of IL15 receptor (IL15R). In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with truncated intracellular domain is fused to IL15 at the C-terminus through a linker, mimicking trans-presentation of IL15, maintaining IL15 membrane-bound, and additionally eliminating cis-presentation and/or any other potential signal transduction pathways mediated by a normal IL15R through its intracellular domain. In other embodiments, IL15Rα is fused to IL15 without an intracellular domain (IL15Δ), as described in International Pub. Nos. WO 2019/191495 and WO 2019/126748, the entire disclosure of each of which is incorporated herein by reference.

In various embodiments, such a truncated construct comprises an amino acid sequence of at least 75%, 80%, 85%, 90%, 95% or 99% identity to SEQ ID NO: 9. In one embodiment of the truncated IL15/IL15Rα, the construct does not comprise the last 4 amino acid residues (KSRQ) of SEQ ID NO: 9, and comprises an amino acid sequence of at least 75%, 80%, 85%, 90%, 95% or 99% identity to SEQ ID NO: 10.

SEQ ID NO: 9
MDWTWILFLVAAATRVHSGIHVFILGCFSAGLPKTEANWVNVISDLKKIEDLIQSMHIDATLYTESDVHP
SCKVTAMKCFLLELQVISLESGDASIHDTVENLIILANNSLSSNGNVTESGCKECEELEEKNIKEFLQSF
VHIVQMFINTSSGGGSGGGGSGGGGSGGGGSGGGSLQITCPPPMSVEHADIWVKSYSLYSRERYICNSGF
KRKAGTSSLTECVLNKATNVAHWTTPSLKCIRDPALVHQRPAPPSTVTTAGVTPQPESLSPSGKEPAASS
PSSNNTAATTAAIVPGSQLMPSKSPSTGTTEISSHESSHGTPSQTTAKNWELTASASHQPPGVYPQGHSD
TTVAISTSTVLLCGLSAVSLLACYLKSRQ
(379 a.a.; signal and linker peptides are underlined)
SEQ ID NO: 10
MDWTWILFLVAAATRVHSGIHVFILGCFSAGLPKTEANWVNVISDLKKIEDLIQSMHIDATLYTESDVHP
SCKVTAMKCFLLELQVISLESGDASIHDTVENLIILANNSLSSNGNVTESGCKECEELEEKNIKEFLQSF
VHIVQMFINTSSGGGSGGGGSGGGGSGGGGSGGGSLQITCPPPMSVEHADIWVKSYSLYSRERYICNSGF
KRKAGTSSLTECVLNKATNVAHWTTPSLKCIRDPALVHQRPAPPSTVTTAGVTPQPESLSPSGKEPAASS
PSSNNTAATTAAIVPGSQLMPSKSPSTGTTEISSHESSHGTPSQTTAKNWELTASASHQPPGVYPQGHSD
TTVAISTSTVLLCGLSAVSLLACYL
(375 a.a .; signal and linker peptides are underlined)

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled in the art understands that the signal peptide and/or linker sequences may be substituted for another sequence without altering the activity of the functional peptide led by the signal peptide or linked by the linker.

In iPSCs and derivative cells therefrom comprising both CAR and exogenous cytokine and/or cytokine receptor signaling (signaling complex or “IL”), the CAR and IL may be expressed in separate constructs, or may be co-expressed in a bi-cistronic construct comprising both CAR and IL, or both hnCD16 and IL. In some further embodiments, the signaling complex can be linked to either the 5′ or the 3′ end of a CAR or hnCD16 expression construct through a self-cleaving 2A coding sequence. As such, an IL signaling complex (e.g., IL15 or IL7 signaling complex) and CAR may be in a single open reading frame (ORF). In one embodiment, the signaling complex is comprised in a CAR-2A-IL or an IL-2A-CAR construct. In one embodiment, the signaling complex is comprised in a hnCD16-2A-IL or an IL-2A-hnCD16 construct. When CAR-2A-IL or IL-2A-CAR, or hnCD16-2A-IL or IL-2A-hnCD16, is expressed, the self-cleaving 2A peptide allows the expressed CAR and IL or hnCD16 and IL, to dissociate, and the dissociated IL can then be presented at the cell surface, with the transmembrane domain anchored in the cell membrane. The CAR-2A-IL or IL-2A-CAR bi-cistronic design, or the hnCD16-2A-IL or IL-2A-hnCD16 bi-cistronic design, allows for coordinated IL signaling complex expression with CAR or hnCD16 both in timing and quantity, and under the same control mechanism that may be chosen to incorporate, for example, an inducible promoter or promoter with temporal or spatial specificity for the expression of the single ORF. Self-cleaving peptides are found in members of the Picornaviridae virus family, including aphthovirus such as foot-and-mouth disease virus (FMDV), equine rhinitis A virus (ERAV), Thosea asigna virus (TaV) and porcine tescho virus-1 (PTV-I) (Donnelly, et al, J. Gen. Virol, 82, 1027-101 (2001); Ryan, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively.

In light of the above, the present application provides an iPSC, an iPS cell line cell, or a population thereof, or a derivative functional cell obtained from differentiating the iPSC, wherein each cell comprises a polynucleotide encoding a CD19 tumor antigen targeting specificity, and wherein the cell comprises polynucleotides encoding a CD19-CAR (chimeric antigen receptor), a 41BB-ADR, a CD16 or a variant thereof that mediates ADCC when in combination with an monoclonal antibody, a CD38 knockout, and an IL15 signaling complex.

In some embodiments, when an anti-CD38 antibody is used to induce CD16 mediated enhanced ADCC, the iPSC and/or its derivative effector cells can target the CD38 expressing (tumor) cells without causing effector cell elimination, i.e., reduction or depletion of CD38 expressing effector cells, thereby increasing persistence and/or survival of the iPSC and its effector cell. In some embodiments, the effector cell has increased persistence and/or survival in vivo in the presence of anti-CD38 therapeutic agents, which may be an anti-CD38 antibody. In addition, since CD38 is upregulated on activated lymphocytes such as T or B cells, an anti-CD38 antibody can be used for lymphodepletion thereby eliminating those activated lymphocytes, overcoming allorejection, and increasing survival and persistency of the CD38 negative effector cells without fratricide in the recipient of the allogeneic effector cell therapy. In some embodiments, the effector cells comprise NK lineage cells. iPSC-derived NK lineage cells comprising CD38 negative and exogenous CD16 or a variant thereof have enhanced cytotoxicity and have reduced NK cell fratricide in the presence of anti-CD38 antibodies.

The iPSC, an iPS cell line cell, or a population thereof, or a derivative functional cell obtained from differentiating the iPSC provided herein, comprising a CD38 knockout and polynucleotides encoding a CD19-CAR (chimeric antigen receptor), a 41BB-ADR, a CD16 or a variant thereof, and an IL15 signaling complex, wherein the exogenous cytokine signaling complex (IL) enables cytokine signaling contributing to cell survival, persistence and/or expansion, wherein the iPSC line is capable of hematopoietic differentiation to produce functional derivative effector cells having improved survival, persistency, expansion, and effector function. In some embodiments, the introduced partial or full peptide of cytokine and/or its respective receptor for cytokine signaling are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, the exogenous cell surface cytokine and/or receptor comprised in the iPSC or derivative cells thereof enables IL15 signaling.

### 5. Chimeric Antigen Receptor (CAR)

In some embodiments, the genomically engineered effector cells provided herein comprise a chimeric antigen receptor (CAR) against CD19. A CAR is a fusion protein generally including an ectodomain that comprises a target binding region (for example, an antigen recognition domain), a transmembrane domain, and an endodomain. In some embodiments, the ectodomain can further include a signal peptide or leader sequence and/or a spacer. In some embodiments, the endodomain can further comprise a signaling peptide that activates the effector cell expressing the CAR. In some embodiments, the signaling peptide of the endodomain (or intracellular domain) comprises a full length or at least a portion of a polypeptide of 2B4, CD2, CD3ζ, CD3ζ1XX, CD8, CD28, CD28H, CD137 (4-1BB), CS1, DAP10, DAP12, DNAM1, FcERIγ, IL2Rγ, IL7R, IL21R, IL2RB (IL15RB), IL7, IL12, IL15, IL21, KIR2DS2, NKp30, NKp44, NKp46, NKG2C, or NKG2D. In one embodiment, the signaling peptide of a CAR comprises an amino acid sequence that has at least about 85%, about 90%, about 95%, about 96%, about 97%, about 98%, or about 99% identity to at least one ITAM (immunoreceptor tyrosine-based activation motif) of CD3ζ. In some embodiments, the CAR comprises an N-terminal signal peptide, such as MALPVTALLLPLALLLHA (SEQ ID NO: 12), or any signal peptide sequence or functional variants thereof known in the art.

In some embodiments, the antigen recognition domain can specifically bind CD19. In some embodiments, the CAR is suitable to activate T, NK or NKT cells expressing the CAR. In some embodiments, the CAR is NK cell specific for comprising NK-specific signaling components. In certain embodiments, the NK cells are derived from iPSCs comprising the CAR.

In various embodiments, the antigen recognition region comprises a murine antibody, a human antibody, a humanized antibody, a camel Ig, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody. In some embodiments an antigen binding domain of a CAR comprises CDR1, CDR2, and CDR3 of a heavy chain (H-CDRs) of an antibody or fragments thereof. In some embodiments, the antigen binding domain of a CAR comprising the H-CDRs of an antibody further comprises the CDRs of a light chain (L-CDRs) of the antibody.

In some embodiments, the scFV is an scFV selected based on having specific binding affinity for a target (e.g., CD19). In some embodiments, the scFV comprises one or more sequences of an antigen-binding fragment of an antibody selected based on having specific binding affinity for the target (e.g., CD19). In some embodiments the antigen binding domain of the CAR comprises a single chain variable fragment (scFV) comprising a heavy chain and a light chain having a sequence identity of at least 85%, 90%, 95%, 99%, 100%, or any percentage in-between, when compared to the exemplary sequences represented by SEQ ID NO: 1 and SEQ ID NO: 2, respectively. In some embodiments, the scFV comprises an amino acid sequence of at least 90% identity to SEQ ID NO: 13 or 14. In some embodiments, the scFV comprises an amino acid sequence of at least 95% identity to SEQ ID NO: 13 or 14. In some embodiments, the scFV comprises the amino acid sequence of SEQ ID NO: 13. In some embodiments, the scFV comprises the amino acid sequence of SEQ ID NO: 14. In some embodiments, the scFV comprises the amino acid sequences of both SEQ ID NO: 13 and 14.

SEQ ID NO: 13
AELVRPGSSVKISCKASGYAFSSYWMNWVKQRPGQGLEWIGQIYPGDG
DTNYNGKFKGQATLTADKSSSTAYMQLSGLTSEDSAVYFCARKTISSV
VDFYF
SEQ ID NO: 14
KFMSTSVGDRVSVTCKASQNVGTNVAWYQQKPGQSPKPLIYSATYRNSG
VPDRFTGSGSGTDFTLTITNVQSKDLADYFCQQYNRYPYTS

In some embodiments, the CAR applicable to the cells provided herein comprises a transmembrane domain derived from NKG2D, a co-stimulatory domain derived from 2B4, and a signaling domain comprising the native or modified CD35, represented by an amino acid sequence of at least about 85%, about 90%, about 95%, about 96%, about 97%, about 98%, or about 99% identity to SEQ ID NO: 15. The CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 16. In some embodiments, the CAR comprises an amino acid sequence with at least 90% sequence identity to SEQ ID NO: 15 or 16. In some embodiments, the CAR comprises an amino acid sequence with at least 95% sequence identity to SEQ ID NO: 15 or 16. In some embodiments, the CAR comprises the amino acid sequence of SEQ ID NO: 15. In some embodiments, the CAR comprises the amino acid sequence of SEQ ID NO: 16.

SEQ ID NO: 15
SNLFVASWIAVMIIFRIGMAVAIFCCFFFPSWRRKRKEKQSETSPKEFLTIYEDVKDLKTRRNHEQEQTF
PGGGSTIYSMIQSQSSAPTSQEPAYTLYSLIQPSRKSGSRKRNHSPSFNSTIYEVIGKSQPKAQNPARLS
RKELENFDVYSRVKFSRSADAPAYKQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
(263 a.a NKG2D TM + 2B4 + CD3ζ)
SEQ ID NO: 16
TTTPAPRPPTPAPTIASQPLSLRPEACRPAAGGAVHTRGLDFACDSNLFVASWIAVMIIFRIGMAVAIFC
CFFFPSWRRKRKEKQSETSPKEFLTIYEDVKDLKTRRNHEQEQTFPGGGSTIYSMIQSQSSAPTSQEPAY
TLYSLIQPSRKSGSRKRNHSPSFNSTIYEVIGKSQPKAQNPARLSRKELENFDVYSRVKFSRSADAPAYK
QGQNQLYNELNLGRREEYDVLDKRRGRDPEMGGKPRRKNPQEGLYNELQKDKMAEAYSEIGMKGERRRGK
GHDGLYQGLSTATKDTYDALHMQALPPR
(308 a.a CD8 hinge + NKG2D TM + 2B4 + CD3ζ)

In some embodiments, the CAR provided herein comprises an amino acid sequence of at least about 85%, about 90%, about 95%, about 96%, about 97%, about 98%, or about 99% identity to SEQ ID NO: 11, wherein the linker in the ectodomain and the spacer between the ectodomain and transmembrane domain may vary in length and sequence. In some embodiments, the CAR comprises an amino acid sequence of at least about 90% identity to SEQ ID NO: 11, wherein the linker in the ectodomain and the spacer between the ectodomain and transmembrane domain may vary in length and sequence. In some embodiments, the CAR comprises an amino acid sequence of at least about 95% identity to SEQ ID NO: 11, wherein the linker in the ectodomain and the spacer between the ectodomain and transmembrane domain may vary in length and sequence. In some embodiments, the CAR comprises the amino acid sequence of SEQ ID NO: 11. In some embodiments, the CAR provided herein recognizes a CD19 antigen, and activates the cell (e.g., an NK cell) in response to binding CD19 of a target cell.

SEQ ID NO. 11
MALPVTALLLPLALLLHAEVKLQQSGAELVRPGSSVKISCKASGYAFSSYWMNWVKQRPGQGLEWIGQIY
PGDGDTNYNGKFKGQATLTADKSSSTAYMQLSGLTSEDSAVYFCARKTISSVVDFYFDYWGQGTTVTVSS
GGGGSGGGGSGGGGSDIELTQSPKFMSTSVGDRVSVTCKASQNVGTNVAWYQQKPGQSPKPLIYSATYRN
SGVPDRFTGSGSGTDFTLTITNVQSKDLADYFCQQYNRYPYTSGGGTKLEIKRAAAPTTTPAPRPPTPAP
TIASQPLSLRPEACRPAAGGAVHTRGLDFACDSNLFVASWIAVMIIFRIGMAVAIFCCFFFPSWRRKRKE
KQSETSPKEFLTIYEDVKDLKTRRNHEQEQTFPGGGSTIYSMIQSQSSAPTSQEPAYTLYSLIQPSRKSG
SRKRNHSPSFNSTIYEVIGKSQPKAQNPARLSRKELENFDVYSRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTAT
KDTYDALHMQALPPR

### 6. Antibodies for Immunotherapy

In some embodiments, in addition to the genomically engineered effector cells as provided herein, additional therapeutic agents comprising an antibody, or an antibody fragment that targets an antigen associated with a condition, a disease, or an indication may be used with these effector cells in a combinational therapy. In some embodiments, the antibody is used in combination with a population of the effector cells described herein by concurrent or consecutive administration to a subject. In other embodiments, such antibody or a fragment thereof may be expressed by the effector cells by genetically engineering an iPSC using an exogenous polynucleotide sequence encoding said antibody or fragment thereof, and directing differentiation of the engineered iPSC. In some embodiments, the effector cell expresses an exogenous CD16 variant, wherein the cytotoxicity of the effector cell is enhanced by the antibody via ADCC. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 antibody (e.g. rituximab, veltuzumab, ofatumumab, ublituximab, ocaratuzumab, obinutuzumab), an anti-EGFR antibody (cetuximab, matuzumab, panitumumab and necitumumab), an anti-HER2 antibody (trastuzumab or biosimilars, pertuzumab, 4B5, ertumaxomab), an anti-PDL1 antibody (avelumab, durvalumab, pembrolizumab, nivolumab, or atezolizumab), a bi-specific antibody targeting EGFR and MET (amivantamab), and their humanized or Fc modified variants or fragments or their functional equivalents and biosimilars. Exemplary trastuzumab biosimilars include, but are not limited to, trastuzumab-anns (Kanjinti™), trastuzumab-dkst (Ogivri®), trastuzumab-qyyp (Trazimera™), trastuzumab-pkrb (Herzuma®), trastuzumab-dttb (Ontruzant®).

In some embodiments, an initial dose of the monoclonal antibody is administered in an effective amount at a starting time prior to a first cycle of administering the iPSC-derived effector cells. In some embodiments, the starting time is about 2-6 days prior to the first cycle of administering the iPSC-derived effector cells. In some embodiments, the antibody for combination treatment is rituximab and wherein the initial dose is a single initial dose of about 300 mg/m² to about 450 mg/m² administered to the subject about 4 days prior to the first cycle of administering the effector cells.

### 7. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Immune checkpoint inhibitors (ICIs) are antagonists capable of reducing checkpoint gene expression or gene products, or deceasing activity of checkpoint molecules, thereby block inhibitory checkpoints, restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern. One aspect of the present application provides a therapeutic approach to overcome ICI resistance by including genomically-engineered functional derivative cells as provided herein in a combination therapy with ICI. In some embodiments, the checkpoint inhibitor is used in combination with a population of the effector cells described herein by concurrent or consecutive administration thereof to a subject. In some other embodiments, the checkpoint inhibitor is expressed by the effector cells by genetically engineering an iPSC using an exogenous polynucleotide sequence encoding said checkpoint inhibitor, or a fragment or variant thereof, and directing differentiation of the engineered iPSC.

In one embodiment of the combination therapy, the derivative cells are NK cells. In addition to exhibiting direct antitumor capacity, the derivative NK cells provided herein have been shown to resist PDL1-PD1 mediated inhibition, and to have the ability to enhance T cell migration, to recruit T cells to the tumor microenvironment, and to augment T cell activation at the tumor site. Therefore, the tumor infiltration of T cells facilitated by the functionally potent genomically-engineered derivative NK cells indicate that said NK cells are capable of synergizing with T cell targeted immunotherapies, including the checkpoint inhibitors, to relieve local immunosuppression and to reduce tumor burden.

Some embodiments of the combination therapy with the provided derivative NK cells comprise at least one checkpoint inhibitor to target at least one checkpoint molecule.

Suitable checkpoint inhibitors for combination therapy with the derivative NK cells as provided herein include, but are not limited to, antagonists of PD1 (Pdcdl, CD279), PDL-1 (CD274), TIM3 (Havcr2), TIGIT (WUCAM and Vstm3), LAG3 (CD223), CTLA4 (CD152), 2B4 (CD244), 4-1BB (CD137), 4-1BBL (CD137L), A2AR, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, a single variable new antigen receptor (VNAR), a shark heavy-chain antibody (Ig NAR), chimeric antibodies, recombinant antibodies, or antibody fragments thereof. Non-limiting examples of antibody fragments include Fab, Fab′, F(ab′)2, F(ab′)3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of pembrolizumab, nivolumab, or atezolizumab (anti-PD1/anti-PDL1 mAb), avelumab (anti-PDL1 mAb), durvalumab (anti-PDL1 mAb), and any derivatives, functional equivalents, or biosimilars thereof.

In some embodiments, ICI for combinational therapy is pembrolizumab administered every three weeks (Q3W) or every six weeks (Q6W) in an amount of about 400 mg. In some embodiments, ICI for combinational therapy is nivolumab administered every two weeks (Q2W) or every four weeks (Q4W) in an amount of: (a) about 240 mg when administered Q2W; or (b) about 480 mg when administered Q4W. In some embodiments, ICI for combinational therapy is atezolizumab administered Q2W, Q3W or Q4W in an amount of: (a) about 840 mg when administered Q2W; (b) about 1200 mg when administered Q3W; or (c) about 1680 mg when administered Q4W.

In some embodiments, an initial dose of the ICI is administered in an effective amount at a starting time prior to a first cycle of administering the iPSC-derived effector cells. In some embodiments, the starting time is about 4-10 days prior to the first cycle of administering the iPSC-derived effector cells. In some embodiments, the ICI for combination treatment is pembrolizumab and is administered in an initial amount of about 200 mg to about 400 mg. In some embodiments, the ICI for combination treatment is nivolumab and is administered in an initial amount of about 240 mg to about 480 mg. In some embodiments, the ICI for combination treatment atezolizumab and is administered in an initial amount of about 840 mg to about 1680 mg.

### II. Therapeutic Use of Derivative Immune Cells for Solid Tumors

Key advancements in cancer immunotherapy include the development of immune checkpoint inhibitor (ICI) monoclonal antibodies (mAbs) that block key inhibitory pathways on T cells and the development of adoptive transfer of immune cells as exemplified by CAR T-cell therapy. Both approaches have led to the development of novel therapeutic regimens that have increased survival in patients with a variety of solid and hematologic tumors. However, the majority of patients will either not respond to treatment or will eventually experience disease relapse. Particularly in solid tumors, the mechanisms of immunotherapy tumor resistance are diverse and include the ability of tumors to form physical and immunologic barriers to immune effector cells such as T cells and NK cells. For example, treatment with anti-programmed cell death-1/programmed death-ligand 1 (anti-PD-1/PD-L1) antibodies has shown promising and durable responses either as monotherapy or in combination with chemotherapy in a growing number of solid tumor indications including, but not limited to, melanoma, renal cell carcinoma, lung cancer, head and neck squamous cell carcinoma, and urothelial carcinoma (UC). However, most patients do not have responses or ultimately experience disease progression on anti-PD-1/PD-L1 therapy. Treatment with mAbs targeting human epidermal growth factor receptor 2 (HER2), epidermal growth factor receptor (EGFR), and/or mesenchymal-epithelial transition (MET) receptor is standard of care for certain tumors that express these targets, but once patients progress beyond approved mAbs for their disease there are limited treatment options available. Thus, therapies that enhance and potentially add anti-tumor activity to these existing mAb therapeutics are an urgent unmet need.

The treatment using the derived hematopoietic lineage cells of embodiments disclosed herein, or the compositions provided herein, could be carried out upon symptom presentation, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; and inhibiting the disease, i.e., arresting its development; or relieving the disease, i.e., causing regression of the disease, or reinduction of disease response to the therapy. Treatment of ongoing disease, where the treatment stabilizes or reduces the undesirable clinical symptoms of the patient, is also of particular interest. In some embodiments, the subject in need of a treatment has a disease, a condition, and/or an injury that can be contained, ameliorated, and/or improved in at least one associated symptom by a cell therapy.

An NK cell product derived from a CD38 knockout iPSC line that comprises polynucleotides encoding a CAR targeting CD19, alloimmune defense receptor (ADR) targeting 4-1BB, hnCD16, and IL-15RF is advanced as an investigational allogeneic NK cell cancer immunotherapy under the Phase 1 study described herein, and is termed FT522. The Phase 1 study described herein evaluates the safety and activity of FT522 in combination with rituximab, with or without conditioning chemotherapy. The features of FT522 as described are designed to provide meaningful clinical benefit of NK-cell therapies, including by obviating the need for conditioning chemotherapy, in participants with R/R BCL in this study.

FT522 as described are designed to mediate and support anti-tumor activity via multiple pathways, including but not limited to: direct cytotoxicity affected via recognition of stress signals and/or by the absence of human leukocyte antigen class I expression; secretion of cytokines/chemokines (interferon-γ, tumor necrosis factor-α, macrophage inflammatory protein 1-α, and -β) that can upregulate death receptors and stress ligands on target cells as well as activate and facilitate an adaptive immune response; antibody-dependent cellular cytotoxicity (ADCC), through which NK cells actively lyse a target cell whose membrane-surface antigens have been bound by specific monoclonal antibodies; targeted cytotoxicity of CD19-expressing cells following recognition by anti-CD19 CAR; targeting of 4-1BB⁺ alloreactive immune cells through engagement of a novel ADR while providing activating CD3ζ signaling. The incorporation of enhanced effector function mediated by CD38 knockout, along with resistance to immune-mediated clearance bestowed by ADR, potentially enables the administration of FT522 without conditioning chemotherapy. These features support the potential for improved clinical activity and reduced toxicity of FT522 for having an active anti-lymphoma therapeutic modality with improved persistence and anti-tumor activity even in the absence of conditioning chemotherapy compared to current-generation cell therapies for the treatment of B-cell malignancies.

There are additional unknown features for human therapeutic use of FT522 that could not be addressed in non-clinical settings. In addition, the nature, frequency, and severity of toxicities, risks and uncertainties extrapolated from other NK cell products and other cell products in general and the known risks of CY/FLU, bendamustine, and rituximab may be investigated, addressed and mitigated in the context of FT522 administration under the study interventions as described herein.

Specific conditions potentially related to engineered cellular immunotherapy products in general include, but are not limited to, new malignancies, new or worsening neurologic disorders, new or worsening autoimmune or rheumatologic disorders, or new hematologic disorders. Participants are undergo safety monitoring during the study, including assessment of the nature, frequency, and severity of adverse events (AEs).

An adverse event (AE) is any untoward medical occurrence in a patient or clinical study subject temporally associated with the use of study treatment, whether or not considered related to the study treatment. An AE can therefore be any unfavorable and unintended sign (including an abnormal laboratory finding), symptom, or disease (new or exacerbated) temporally associated with the use of a medicinal (investigational) product, whether or not related to the medicinal (investigational) product.

Events meeting the definition of AE include, but are not limited to, the following: (i) Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator (i.e., not related to progression of underlying disease). (ii) Exacerbation of a chronic or intermittent pre-existing condition including either an increase in frequency and/or intensity of the condition. (iii) New conditions detected or diagnosed after study treatment administration even though it may have been present before the start of the study. (iv) Signs, symptoms, or the clinical sequelae of a suspected drug-drug interaction. (v) Signs, symptoms, or the clinical sequelae of a suspected overdose of either study treatment(s) or a concomitant medication. Overdose per se is not considered an AE/SAE (serious adverse event).

Acute allergic/infusion reactions may occur with any treatment, including with the use of CY, FLU, bendamustine, and mAbs. Participants are closely monitored for the occurrence of acute allergic/anaphylactoid infusion reactions such as rigors and chills, rash, urticaria, hypotension, dyspnea, and angioedema during and following completion of the infusion. Acute allergic/infusion reactions may also be a manifestation of immunogenicity of an allogeneic cell product.

Evidence of FT522 immunogenicity and its clinical impact is monitored during the study, given that potential FT522-induced immune response may manifest only through laboratory assessments, or may manifest clinically, e.g., as infusion-related reactions with varying degrees of severity. Adverse events arising from FT522 immunogenicity is managed per institutional practice.

FT522 is formulated in DMSO to enable cryopreservation. DMSO side effects and symptoms are generally associated with histamine release and include coughing, flushing, rash, chest tightness and wheezing, nausea and vomiting, and cardiovascular instability. Slowing the rate of infusion, medicating with antihistamines, and treating symptoms are practices followed in the study.

FT522 is a cell therapy of human origin. During processing, the cells are in contact with reagents of animal origin, and FT522 has a final formulation which contains albumin (human). As with any product of human and/or animal origin, transmission of infectious disease and/or disease agents by known or unknown agents may occur. FT522 has been extensively tested to minimize the potential risk of disease transmission. However, the study may detect and address disease transmission by some infectious agents imposing risks that do not have routine tests to predict or prevent other than when manifested during human study.

Cytokine Release Syndrome (CRS) is defined as a supraphysiologic response following any immune therapy that results in the activation or engagement of endogenous or infused immune effector cells. Clinical manifestations of CRS include cardiac, gastrointestinal, hepatic, coagulation, renal, respiratory, skin, and constitutional (fever, rigors, headaches, malaise, fatigue, arthralgia, nausea, and vomiting) signs and symptoms. Treatment-emergent AEs that may be attributed at least in part to CRS include fever, febrile neutropenia, hypotension, acute vascular leak syndrome, renal failure, hypoxia, and pleural effusion. While CRS is a clearly defined syndrome with CAR T-cell therapy, it has generally not been observed as a toxicity associated with NK-cell therapies unless administered with systemic cytokines that may independently drive the proliferation and activation of CD8+ T cells, e.g., exogenous IL-15. Clinical symptoms as noted above that are not considered related to FT522 are not reported as CRS.

Tumor Lysis Syndrome (TLS) is a possible fatal risk associated with anti-tumor therapy in both hematologic and solid tumors, especially with large tumor burden. TLS has been reported to occur within 7 days following chemotherapy across various solid tumor settings, with 10 published reports of TLS cases in patients with gynecological cancer. One case of fatal metabolic syndrome compatible with TLS was reported following NK-cell therapy in a patient with ovarian cancer 5 days after receiving CY. TLS symptoms include nausea, vomiting, diarrhea, muscle cramps or twitches, weakness, numbness or tingling, fatigue, decreased urination, irregular heart rate, restlessness, irritability, delirium, hallucinations, and seizures. TLS is comprised of abnormal laboratory changes that include hyperuricemia, hyperkalemia, hyperphosphatemia, and hypocalcemia. Prophylaxis for and management of TLS is done in accordance with standard institutional practice.

Neurologic toxicities arising as a result of immune therapies has been termed Immune Effector Cell-Associated Neurotoxicity Syndromes, or ICANS, defined as a disorder characterized by a pathologic process involving the CNS following any immune therapy that results in the activation or engagement of endogenous or infused immune effector cells. ICANS has been reported with CAR T-cell therapy and bispecific antibodies such as blinatumomab (Blincyto® USPI). The exact mechanism of toxicity in these settings is not known, but has generally improved with treatment discontinuations and corticosteroids. Central nervous system toxicities following CD19 CAR T-cell therapy are characterized by encephalopathy, confusion, delirium, aphasia, obtundation, and seizures (Kymriah® USPI; Yescarta® USPI). Cases of cerebral edema have also been reported in CAR-T therapies. While ICANS is a clearly defined syndrome associated with CAR T-cell-based therapies, it is rare and generally not believed to be a toxicity associated with NK-cell therapies, but observation is carried out.

Because FT522 is an allogeneic immune effector cell product, there is a potential risk of Acute Graft-versus-Host Disease (GvHD) even though allogeneic NK-cell therapies have not been associated with such risk. Acute GvHD assessments are performed with assignment of the overall severity based on the CIBMTR acute GvHD grading scale, and the management of GvHD is done in accordance with local institutional practice.

In addition, FT522 is considered a xenotransplantation product because it comes in contact with cells of animal origin (mouse cells) during the manufacturing process, i.e., the cells of animal origin serve as an ancillary material in the manufacture of drug product but are not intended to be in the drug product. Risks of receiving a xenotransplantation product may include, but are not limited to, developing infections from agents that may be associated with cells of animal origin, transmitting these infectious agents to others, and the growth of tumors. The animal cells, which come into contact with the FT522 cells during the manufacturing process, originate from a master cell bank that has been extensively tested to reduce these risks. However, these tests do not completely eliminate this risk. Patients are monitored for clinical manifestations of xenotransplantation products throughout the course of the study, including long-term follow-up, in case of rare associated condition or illness develops.

Some adoptive cell therapies delivered with supportive medications, such as Cyclophosphamide (CY) and Fludarabine (FLU) for conditioning, have been reported to cause myelosuppression (neutropenia and/or thrombocytopenia), immunosuppression, infections, leukopenia, anemia, and in some cases, bone marrow failure. Hematologic cytopenias could be further compounded by other factors such as underlying disease, concurrent illnesses, and concomitant medications. Close monitoring of complete blood count for the development of cytopenias and infections is undertaken. Management of cytopenias and infections, including transfusion support, antimicrobial prophylaxis, and use of growth factors, is done in accordance with standard institutional practice.

Warnings and precautions related to cyclophosphamide (CY) include: myelosuppression, immunosuppression, bone marrow failure, and infections; urinary tract and renal toxicity including hemorrhagic cystitis, pyelitis, ureteritis, and hematuria; cardiotoxicity including myocarditis, myopericarditis, pericardial effusion, arrythmias, and congestive heart failure; pulmonary toxicity including pneumonitis, pulmonary fibrosis, and pulmonary veno-occlusive disease leading to respiratory failure; secondary malignancies; veno-occlusive liver disease; and embryo-fetal toxicity. Adverse reactions reported most often include neutropenia, febrile neutropenia, fever, alopecia, nausea, vomiting, and diarrhea. Dose modifications due to toxicity are taken into consideration in the study.

Warnings and precautions related to fludarabine (FLU) include severe bone marrow suppression, notably anemia, thrombocytopenia, and neutropenia; transfusion-associated GvHD; severe CNS toxicity; infections; renal insufficiency; TLS; and embryo-fetal toxicity. Severe CNS toxicity was observed in patients treated at FLU doses of 96 mg/m² for 5 to 7 days. This toxicity was observed in ≤0.2% of patients treated at FLU doses of 25 mg/m². Adverse reactions occurring in >30% of participants treated with FLU include myelosuppression (neutropenia, thrombocytopenia, and anemia), fever, infection, nausea and vomiting, fatigue, anorexia, cough, and weakness.

Warnings and precautions related to bendamustine include myelosuppression, infections, infusion reactions and anaphylaxis TLS; skin reactions including rash, toxic skin reactions (such as Stevens-Johnson syndrome and toxic epidermal necrolysis), and bullous exanthema; other malignancies; and fetal harm.

Risks associated with rituximab or biosimilars (for example, Truxima USPI; Ruxience USPI; Riabni USPI) include fatal infusion reactions; severe mucocutaneous reactions HBV reactivation that may result in fulminant hepatitis, hepatic failure; and progressive multifocal leukoencephalopathy (PML). Depending on the toxicity, rituximab should be withheld or discontinued per the current local prescribing information.

Additional adverse events observed with rituximab include TLS, infections, cardiac adverse reactions, renal toxicity, bowel obstruction and perforation, embryo-fetal toxicity. The most common adverse reactions (nearly 25%) in individuals with NHL are infusion reactions, fever, lymphopenia, chills, infection, and asthenia.

In addition to the known and potential risks of FT522, CY, FLU, bendamustine, and mAbs, additional risks of the combination treatment due to drug interaction including, but not limited to, increased frequency and/or severity of risks known to the mAb may occur. Therefore evidences of toxicities of approved mAbs whose frequency and severity may be affected by combination treatment with FT522 are collected and dealt with should such risks present during the study.

### EXAMPLES

The following examples are offered by way of illustration and not by way of limitation.

### Example 1—Study Treatments

FT522 is an off-the-shelf NK-cell product candidate that is manufactured from a clonal master iPSC line that has the potential to address the shortcomings of current adoptive cell therapy. The functional attributes of FT522 include expression of the hnCD16 Fc receptor, IL-15RF, and an anti-CD19 CAR (SEQ ID NO. 11), in addition to a CD38 knockout. The study was designed to evaluate the safety of FT522 and determine the recommended Phase 2 dose (RP2D) and schedule for FT522 in combination with rituximab, with or without conditioning chemotherapy, in participants with relapsed and/or refractory B-cell lymphoma

(R/R BCL).

SEQ ID NO. 11
MALPVTALLLPLALLLHAEVKLQQSGAELVRPGSSVKISCKASGYAFSSYWMNWVKQRPGQGLEWIGQIY
PGDGDTNYNGKFKGQATLTADKSSSTAYMQLSGLTSEDSAVYFCARKTISSVVDFYFDYWGQGTTVTVSS
GGGGSGGGGSGGGGSDIELTQSPKFMSTSVGDRVSVTCKASQNVGTNVAWYQQKPGQSPKPLIYSATYRN
SGVPDRFTGSGSGTDFTLTITNVQSKDLADYFCQQYNRYPYTSGGGTKLEIKRAAAPTTTPAPRPPTPAP
TIASQPLSLRPEACRPAAGGAVHTRGLDFACDSNLFVASWIAVMIIFRIGMAVAIFCCFFFPSWRRKRKE
KQSETSPKEFLTIYEDVKDLKTRRNHEQEQTFPGGGSTIYSMIQSQSSAPTSQEPAYTLYSLIQPSRKSG
SRKRNHSPSFNSTIYEVIGKSQPKAQNPARLSRKELENFDVYSRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTAT
KDTYDALHMQALPPR

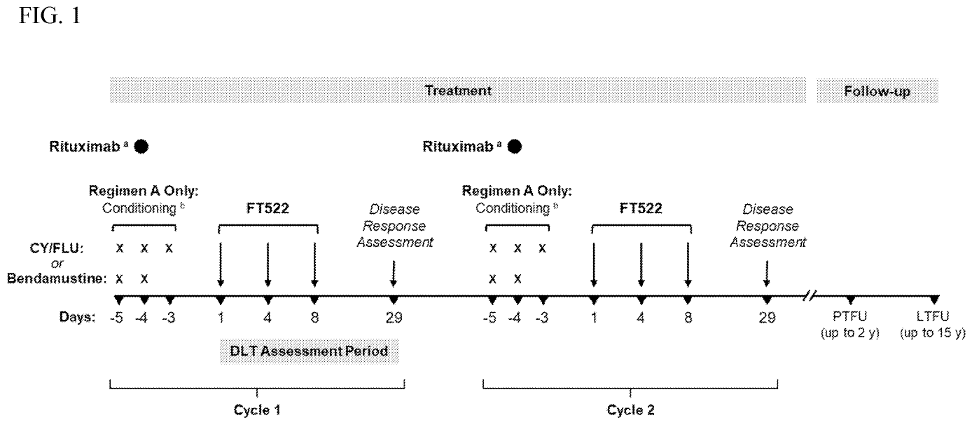

For Regimen A (FIG. 1), FT522 is administered in combination with rituximab (or a rituximab biosimilar approved by a local health authority) with conditioning chemotherapy. The treatment comprises up to 2 treatment cycles, each cycle consisting of conditioning chemotherapy and rituximab followed by 3 doses of FT522 (administered on Days 1, 4, and 8).

The purpose of conditioning prior to the administration of FT522 is to create an immune environment amenable to FT522 persistence and expansion. This is accomplished by promoting homeostatic proliferation of FT522 as well as eliminating regulatory immune cells and other competing elements of the immune system that compete for homeostatic cytokines.

Bendamustine is an alkylating agent with a well-characterized and relatively favorable safety profile as a component of immunochemotherapy regimens, and may be administered in multiple treatment cycles, for the treatment of B-cell malignancies (Treanda® USPI).

As described above, current dosing regimens in which cellular immunotherapies are administered after conditioning chemotherapy were developed in the context of CAR T-cell therapy. While the clinical benefit of CAR T-cell therapy has been well documented, the toxicity of conditioning chemotherapy is significant, particularly in participants who have received multiple prior therapies, such as those enrolling into this study. High-grade cytopenias are common and, while usually reversible, may persist for longer periods in heavily pretreated patients. For example, LTFU of participants treated with axicabtagene ciloleucel demonstrated that 17% of the treated participants had Grade >=3 cytopenias for more than 3 months after treatment (Locke et al., 2019). Clinical data from Study FT596-101 indicate that the most common Grade >=3 AEs were associated with hematologic cytopenias, suggesting at least a contributory role for conditioning chemotherapy (Bachanova et al., 2021).

For regimen B, FT522 is administered in combination with rituximab (or a rituximab biosimilar approved by a local health authority) without conditioning chemotherapy. The treatment comprises up to 2 treatment cycles, each cycle consisting of rituximab followed by 3 doses of FT522 (administered on Days 1, 4, and 8).

The justification for conducting dose escalation with FT522 in combination with rituximab but without conditioning chemotherapy is to fully assess the safety, PK, and clinical activity of a CAR-NK cell carrying the ADR, which is hypothesized to prevent clearance by alloreactive T cells and therefore allow sustained persistence and anti-tumor activity. The presence of the ADR is hypothesized to reduce or eliminate the need for conditioning chemotherapy.

The FT522 dosing schedule in this study, whereby FT522 is administered on Day 1, Day 4, and Day 8 of a treatment cycle, centers on the opportunity to drive deeper and/or more durable responses and improve clinical outcomes. Similar to the determination of FT522 starting dose there is currently no established approach to the design of nonclinical studies to support dosing schedules in CAR-iNK cell therapy dose-escalation studies.

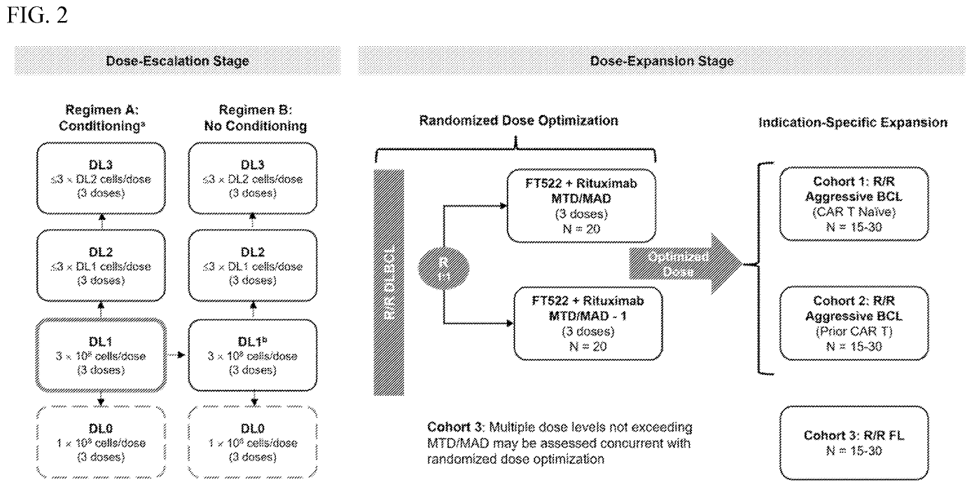

FIG. 2 describes the dose-escalation and dose-expansion schema. The objective of the dose-escalation stage is to determine dose levels of FT522 not exceeding the Maximum tolerated dose (MTD) appropriate for further assessment in dose expansion to determine the RP2D. Planned starting FT522 dose levels in dose escalation are as follows—DL0: 1×10⁸ cells per dose (to be assessed if DL1 is not tolerable or exceeds the MTD); DL1: 3×10⁸ cells per dose. Note that dosing is based on CD19 CAR expression, where ≥80% of administered FT522 viable cells express CD19 CAR.

If the initial dose level does not exceed the MTD, successive dose levels to be tested will not exceed 3 times (±15%) the highest cleared dose level. In the event that an evaluated dose level exceeds the MTD, intermediate dose levels can be explored. In the absence of dose limiting toxicities, dose levels to be tested may be based on observed safety, tolerability, activity, and pharmacokinetic (PK) and pharmacodynamic data.

When dose escalation/de-escalation is conducted, an mTPI algorithm (Ji et al., 2010) is utilized with a target dose-limiting toxicity (DLT) rate of 25% and an equivalence interval of (20%, 30%).

The progression of FT522 dose escalation is described herein. Dose escalation initiates with Regimen A, Dose Level (DL) 1. The initiation of Regimen B dose escalation is contingent on the clearance of at least one dose level in Regimen A and may proceed starting at the highest cleared dose level in Regimen A. For example, Regimen B dose escalation beginning at DL1 may be initiated following clearance of Regimen A DL1.

Dose escalation of Regimen A and Regimen B is conducted independently until the MTD/MAD (the maximum tolerated dose/the maximum assessed dose) is identified for each regimen. Under situations where continued dose escalation is preferentially conducted for Regimen A or Regimen B only, dose escalation in the other regimen proceeds as described herein. If dose escalation is initially conducted for Regimen A only, initiation of Regimen B dose escalation proceeds starting at the highest cleared dose level in Regimen A. If Regimen B dose escalation is conducted in absence of concurrent Regimen A dose escalation, resumption of Regimen A dose escalation may be initiated at the highest cleared dose level in Regimen B. For Regimen A and Regimen B, if DL1 is not tolerable or exceeds the MTD, Regimen A or Regimen B DL0 may be explored.

A DLT is defined as any AE that is at least possibly related to FT522 following the first infusion of FT522 on Day 1 through the end of the DLT assessment period on Day 29, with extension of the DLT assessment period beyond Cycle 1 Day 29 to allow for AE recovery. Grading of AEs and therefore of DLTs are based on the National Cancer Institute Common Terminology Criteria for Adverse Events, Version 5.0 (NCI CTCAE, v5.0) or the American Society for Transplantation and Cellular Therapy (ASTCT) Consensus Grading Guidelines for Cytokine Release Syndrome and Neurological Toxicity Associated with Immune Effector Cells.

The objectives of the dose-expansion stage include further assess safety and tolerability and to identify activity signals to guide and support future development of FT522 in BCL. Dose expansion could occur independently by regimen, dose/dosing schedule, and indication. The dose for dose expansion for each regimen is determined based on the clinical and available PK and pharmacodynamic data from the respective dose-escalation stage and will not exceed the MTD or MAD. Dose expansion cohort(s) enrolls when a given dose level is cleared in dose escalation. One or more dose levels that have cleared DLT assessment in dose escalation in Regimen A and/or Regimen B are evaluated in dose expansion to further define clinical activity. Combined safety and efficacy analyses from both dose escalation and dose expansion are used to determine the RP2D for each regimen.

Dose expansion includes randomized dose-optimization cohorts (n=20 cohort) of FT522 in R/R diffuse large B-cell lymphoma (DLBCL) to compare 2 different dose levels for Regimen A and/or Regimen B not exceeding the MTD/MAD to more robustly evaluate safety, activity, and PK at the different doses.

In addition to the randomized dose-optimization cohort, the following optional non-randomized indication-specific dose-expansion cohorts of up to 30 participants per cohort are opened at a dose level for Regimen A and/or Regimen B not exceeding the MTD/MAD. The Cohort 1 includes patients with R/R aggressive BCL (including DLBCL that is not otherwise specified, HGBCL, transformed indolent NHL, primary mediastinal BCL, and Grade 3B FL) who have received at least 2 prior therapies (including anti-CD20 and anthracycline) and who have received prior CAR T-cell therapy. The Cohort 2 includes participants with R/R aggressive BCL (including DLBCL that is not otherwise specified, HGBCL, transformed indolent NHL, primary mediastinal BCL, and Grade 3B FL) who have received at least 2 prior therapies (including anti-CD20 and anthracycline) and who have not received prior CAR T-cell therapy. The Cohort 3 includes participants with R/R low-grade FL (Grades 1, 2, or 3A) following at least 2 prior therapies including at least one therapy containing an anti-CD20 mAb.

The current study allows protocol amendments to enable enrollment of additional dose-expansion cohorts of up to 30 participants per cohort in order to evaluate FT522 in specific BCL patient populations defined by baseline clinical characteristics, e.g., prior therapies, age, tolerability to standard immunochemotherapy, or to more fully characterize safety/tolerability at a given dose level, including evaluation of alternate doses and schedules of FT522 to optimize safety and tolerability, provided the dose of FT522 does not exceed the MTD or MAD.

### Example 2—FT522 Cycle 2 Treatment

After the above Cycle 1 treatment, continued treatment with a second cycle (Cycle 2) comprising conditioning chemotherapy, if applicable, FT522, and rituximab with the same dose and schedule of FT522 used in Cycle 1, is an option following the criteria described herein:
(1) The participant did not experience a DLT.
(2) A participant who developed tumor lysis syndrome (TLS) that met the DLT criteria (i.e., considered to be at least possibly related to FT522).
(3) Resolution of treatment-related neutropenia and thrombocytopenia to Grade <=2 or baseline.
(4) Resolution of treatment-related non-hematologic AEs that are considered clinically significant to Grade 1 or baseline.

Evidence of ongoing clinical benefit including, but not limited to: absence of signs and symptoms (including worsening laboratory values) that indicate disease progression. No decline in Eastern Cooperative Oncology Group (ECOG) Performance Status. No clear evidence of progressive disease (PD) based on Lugano 2014 classification. In cases of pseudoprogression, where there is manifestation of apparent increase of existing tumor masses or the appearance of new tumor lesions that are caused by the influx of immune cells into tumor sites, the participant is allowed to continue study intervention provided there is an absence of signs and symptoms (including worsening of laboratory values) indicating clinical disease progression and an absence of apparent tumor progression at critical anatomical sites where the risk of compromised organ function is considered unacceptable.

The rationale of a multi-dose treatment schedule of FT522 is to maximize clinical activity. Subsequent FT522 cycles also provide insight into the overall duration of treatment that is safe and tolerable as well as the potential clinical benefit of a longer duration of treatment.

Although the presence of the ADR in FT522 is hypothesized to reduce the need for conditioning chemotherapy, it is plausible that optimal activity will in fact require administration of such conditioning. In this case, the PK window following a single round of conditioning chemotherapy may allow only a limited number of FT522 doses to be administered. Additional rounds of conditioning chemotherapy may be required to maximize clinical benefit.

For participants receiving treatment with an additional cycle, study intervention and clinical assessments, including response assessments, will be performed following the same schedule as Cycle 1 with the exception that an exploratory biopsy between Days 2 and 8 is not required. The dose of conditioning chemotherapy (if relevant) may be reduced. The schedule of blood draws for serum biomarkers, immune monitoring, PK, etc., will also be the same as for Cycle 1.

### Example 3—Study Population for Each Study Cohort

This study evaluates the clinical activity of FT522 in participants with R/R BCL that, despite improvements in outcomes with evolving therapies, remains a disease with areas of significant unmet medical need. Given that FT522 expresses hnCD16, an objective of this study is to assess whether the combination of FT522 with rituximab exhibits clinical activity that exceeds what has been observed with rituximab alone. In addition, FT522 contains a CD19 CAR. CD19 is a B-cell lineage specific surface protein against which CAR T-cell therapies have demonstrated clinical benefit (Kymriah® USPI; Yescarta® USPI; Breyanzi® USPI).

Eligible participants with diagnosis of BCL meet all of the criteria as described herein. First, the patient must have histologically documented lymphomas that are expected to express CD19 and CD20, which include Grades 1 to 3B follicular lymphoma (FL), transformed indolent non-Hodgkin lymphoma (NHL), DLBCL (not otherwise specified), high-grade BCL, and primary mediastinal BCL. Secondly, the patient having the R/R disease following at least 1 prior systemic regimen containing an anti-CD20 mAb has no available curative treatment options. Third, the patient has evaluable F-fluorodeoxyglucose (FDG)-avid disease, or measurable disease defined by at least one bi-dimensionally measurable lesion, e.g., nodal lesion >1.5 cm in longest dimension or extra-nodal lesion >1.0 cm in longest dimension by computed tomography (CT) scan. Any lesion that has been irradiated within 28 days of Day 1 is not considered measurable. For indication-specific dose-expansion cohorts, additional specific inclusion criteria are also provided for those having the R/R disease. For Cohort 1: participants are those with aggressive BCL (including DLBCL (not otherwise specified), HGBCL, transformed indolent NHL, primary mediastinal BCL, and Grade 3B FL who have received at least 2 prior therapies (including anti-CD20 and anthracycline) and who have received prior CAR T-cell therapy. For Cohort 2: Participants are those with aggressive BCL (including DLBCL (not otherwise specified), HGBCL, transformed indolent NHL, primary mediastinal BCL, and Grade 3B FL) who have received at least 2 prior therapies (including anti-CD20 and anthracycline) and who have NOT received prior CAR T-cell therapy. For Cohort 3: participants are those with R/R Grade 1, 2, or 3A FL following at least 2 prior therapies including at least one therapy containing an anti-CD20 mAb.

Currently, the first line treatments include, but are not limited to, drugs such as rituximab, obinutuzumab (Gazyva), chlorambucil, fludarabine, bendamustine, Hyper-CVAD (cyclophosphamide, vincristine, doxorubicin (Adriamycin), and dexamethasone, alternating with high-dose methotrexate plus cytarabine), R-CHOP (rituximab, cyclophosphamide, doxorubicin, vincristine, and prednisone, alternating with rituximab and cytarabine), or RDHAP (rituximab, dexamethasone, cytarabine, and cisplatin). Later lines of treatments include, but are not limited to, drugs such as ibrutinib (Imbruvica), acalabrutinib (Calquence), zanubrutinib (Brukinsa), pirtobrutinib (Jaypirca), bortezomib (Velcade), or venetoclax (Venclexta), lenalidomide (Revlimid), or combinations thereof with or without rituximab; and CAR T-cell therapy.

### Example 4—Study Interventions and Concomitant Therapy

Study interventions are all pre-specified, investigational and non-investigational medicinal products intended to be administered to the study participants during the study conduct, which include FT522, designated as Investigational Medicinal Product (IMP); and authorized auxiliary medicinal product (AxMP) that are non-IMP, such as rituximab (or a rituximab biosimilar approved by a local health authority), cyclophosphamide (CY), fludarabine (FLU), and bendamustine. an overview of study interventions administered is provided in Table 1.

FT522. FT522 drug product comprises allogeneic NK cells, derived from a CD38 knockout iPSC line, that express a CAR targeting CD19, ADR targeting 4-1BB, hnCD16, and IL-15RF. FT522 cells are suspended in infusion medium containing albumin (human) and DMSO. FT522 is provided in a cryopreserved bag and thawed at the site of administration. FT522 is administered as an IV infusion via gravity at planned dose levels using an IV administration set with an in-line filter. Prior to administration of FT522, participants are pre-medicated with acetaminophen 650 mg orally (PO) and diphenhydramine 25 to 50 mg PO or IV before and 4 to 6 hours after administration. Corticosteroids must not be used as pre-medication for FT522.

Conditioning Chemotherapy. Cyclophosphamide: Immediately prior to CY administration, participants receive 500 mL of IV normal saline, and optionally receive additional IV normal saline following CY administration as needed. Dose adjustments for weight/creatinine may be made. CY is administered as an IV infusion at a dose of 500 mg/m² for 3 consecutive days on Day −5, Day −4, and Day −3 of the treatment cycle. CY dosing is calculated based on actual body weight (ABW). If ABW is >150% of the ideal body weight (IBW) then the dose is computed using adjusted body weight as follows:

$$Adjusted body weight = IBW + 0.5 ( ABW - IBW )$$

TABLE 1
Study Interventions

| | | |
|---|---|---|
| Study | Route of | |
| Intervention | Administration | Dose and Schedule |

Conditioning Chemotherapy (Regimen A Only)

| | | |
|---|---|---|
| Cyclophosphamide | IV infusion | 500 mg/m²; Days −5, −4, and −3 of each cycle^{a} |
| Fludarabine | IV infusion | 30 mg/m²; Days −5, −4, and −3 of each cycle^{a} |

OR

| | | |
|---|---|---|
| Bendamustine | IV infusion | 90 mg/m²; Days −5 and −4 of each cycle^{a} |

Rituximab and FT522 (Regimen A and Regimen B)

| | | |
|---|---|---|
| Rituximab^{b} | IV infusion | 375 mg/m²; Day −4 of each cycle |
| FT522 | IV infusion | Dose per cohort is to be determined; Days 1, |
| | | 4, and 8 of each cycle |

^{a}Participants enrolled to Regimen A receive CY/FLU or bendamustine. Dose adjustments may be undertaken for Cycle 2 conditioning as outlined herein.
^{b}A rituximab biosimilar that has been approved by a local health authority may be used.

Fludarabine: FLU is administered as an IV infusion at a dose of 30 mg/m² for 3 consecutive days on Day −5, Day −4, and Day −3 (with a window of +1 day, i.e., on Day −4, −3, −2) of the treatment cycle. Dose adjustments for weight and/or renal function (e.g., as assessed by creatinine clearance) may be made.

Bendamustine: Bendamustine is administered as an IV infusion at a dose of 90 mg/m² per institutional standard of care for 2 consecutive days on Days −5 and −4 (with a window of +1 day).

Rituximab: Rituximab is administered as an IV infusion at a dose of 375 mg/m² on Day −4 of the treatment cycle (with a window of +1 day). Pre-medications for infusion-related reactions may be administered based on need. Alternatively, a rituximab biosimilar such as rituximab-abbs [Truxima®], rituximab-pvvr [Ruxience™ ], or rituximab-arrx [Riabni™] may be used.

Concomitant therapy. Concomitant therapy refers to any medication, including but not limited to prescription drugs, over-the-counter drugs, vaccines, herbal or homeopathic remedies, nutritional supplements that may be used by a participant in addition to the study intervention as described herein. Permitted therapies include supportive care therapies, including but not limited to antibiotics, analgesics, transfusions, growth factors, and irradiated blood products that are used to minimize the risk of transfusion-associated GvHD. Permitted therapies must not be those that are specifically prohibited during the study. Prohibited therapies include systemic corticosteroids, which should be avoided during the treatment cycle, unless absolutely required, because they may inhibit NK-cell function. Because of their deleterious effect on NK cell-based therapy, corticosteroids as pre-medication for CY, FLU, and bendamustine should be avoided and should not be administered within 24 hours before or after FT522 administration. Also prohibited are glucocorticoids as pre-medication for FT522. Intravenous glucocorticoids as pre-medication for CY, FLU, bendamustine, and/or rituximab may be administered, in which case, methylprednisolone is preferred for its shorter half-life, whereas long-acting corticosteroids, such as dexamethasone, should not be used. In addition, any antineoplastic agent for therapeutic intent other than the present study intervention(s) is prohibited, which includes, and is not limited to, radiation therapy following the initiation of study intervention or corticosteroids administered specifically as treatment for lymphoma.

### Example 5—Dose and Schedule Modifications of the Study

Dose and Schedule Modifications for CY/FLU. Dose and schedule modifications may be made for CY/FLU. For participants with moderate impairment of renal function (creatinine clearance of 30-70 mL/min/1.73 m²), the FLU dose should be reduced by 20%. FLU is not administered for participants with severely impaired renal function (creatinine clearance <30 mL/min/1.73 m²).

For participants who complete the first treatment cycle (Cycle 1), the determination of the need for dose reduction is based on complete blood count (CBC) on Day 29 and/or the presence of treatment-related AEs during Cycle 1.

If Grade >=3 AEs of neutropenia and thrombocytopenia that are considered at least possibly related to CY/FLU, do not recover to Grade 2 or baseline by Day 29, and/or for participants who experience significant treatment-related AEs (i.e., serious infections requiring hospitalization), the following dose modifications are made: reduce the daily dose of CY to 300 mg/m² on Days −5, −4, and −3; and reduce the daily dose of FLU to 25 mg/mg² on Days −5, −4, and −3 in Cycle 2.

If additional CY/FLU is not in the participant's best interest (e.g., based on prior therapies or observed toxicities), CY/FLU may be omitted.

Dose and Schedule Modifications for Bendamustine. Dose and schedule modifications may be made for bendamustine. Bendamustine is not administered in participants who have renal or hepatic impairment as defined by:

$$Creatinine clearance < 40 mL / min$$

AST or ALT that is 2.5 to 10×ULN, and total bilirubin that is 1.5 to 3×ULN; or total bilirubin >3×ULN

Bendamustine administration is delayed in the event of Grade 4 hematologic toxicity or clinically significant Grade >=2 non-hematologic toxicity. Once non-hematologic toxicity has recovered to Grade 1 or baseline grade, whichever is higher, and/or blood counts have improved, e.g., ANC ³1000/μL, platelets ³75000/μL or baseline grade, whichever is higher, bendamustine may be administered. In addition, the following dose modifications are made for toxicities attributable to bendamustine. For example, for Grade 4 hematologic toxicity, bendamustine dose may be reduced to 60 mg/m² on Days −5 and −4 in subsequent treatment cycles. If Grade 4 toxicity recurs, bendamustine dose may be further reduced to 40 mg/m² on Day −5 and −4 in subsequent treatment cycles. If Grade 4 toxicity recurs, discontinue bendamustine administration. For non-hematologic toxicity of Grade >=3, reduce bendamustine is reduced to 60 mg/m² on Days −5 and −4 in subsequent treatment cycles. If Grade >=3 toxicity recurs, reduce bendamustine dose to 40 mg/m² on Day −5 and −4 in subsequent treatment cycles. If Grade >=3 recurs, discontinue bendamustine administration. Additional bendamustine administration may be omitted based on patient's prior therapies or observed toxicities.

Dose and Schedule Modifications for FT522 Within a Cycle. Dose and schedule modifications of FT522 within a cycle is allowed under following circumstances: (1) if a DLT is observed, i.e., at least possibly related to FT522, then no further FT522 infusions is administered; (2) if a Grade 3 non-hematologic AE that is not a DLT is observed, is considered related to FT522, and is ongoing at the time of the subsequent scheduled FT522 infusion, FT522 infusion is delayed until resolution of the AE to no more than Grade 1 or baseline, at which time the FT522 dose will be reduced to at least one lower dose level; (3) if recovery to Grade ≤2 or baseline is not observed by the next scheduled FT522 dose, that scheduled FT522 infusion is skipped; (4) in the setting of a Grade 3 or 4 hematologic AE occurring during a cycle, FT522 dosing continues according to the schedule and without dose modifications unless the patient becomes clinically unstable.

### Example 6—Tumor Response Assessment and Efficacy Analyses

Approximately 36 participants per regimen are enrolled in the dose-escalation stage for Regimens A and B. For the randomized assessment of FT522 in R/R DLBCL at 2 different dose levels for Regimen A and/or Regimen B during dose expansion, the sample size of 20 participants per arm is determined to allow for numerical differentiation between 2 dose levels without formal hypothesis testing. A sample size of 30 participants for each indication-specific dose expansion cohort for Regimen A and/or Regimen B provides an opportunity to identify early signs of promising clinical activity for subsequent Phase 2 expansion. Table 2 displays the exact 95% CI for the true response rate of 40%, 50%, 60%, and 70%.

TABLE 2
True Response Rate and Exact 95% Confidence Interval

| Dose-Expansion | True | Lower Limit of | Upper Limit of |
|---|---|---|---|
| Cohort | Response | Exact 95% | Exact 95% |
| Sample Size | Rate | Confidence Interval | Confidence Interval |
| 20 participants | 40% | 19.1% | 64.0% |
| | 50% | 27.2% | 72.8% |
| | 60% | 36.1% | 80.9% |
| | 70% | 45.7% | 88.1% |
| 30 participants | 40% | 22.7% | 59.4% |
| | 50% | 31.3% | 68.7% |
| | 60% | 40.6% | 77.3% |
| | 70% | 50.6% | 85.3% |

Target lesion responses are categorized as complete response (CR), partial response (PR), stable disease (SD), or progressive disease (PD). For non-target lesions in the absence of quantitative measurement, responses are characterized as follows: CR, non-CR/non-PD (corresponding to PR/SD), or PD. The best overall response (BOR) is determined based on an overall evaluation of target lesions, non-target lesions, and new lesions. The BOR is defined as the best overall response recorded from the start of study intervention until disease progression or relapse. The baseline measurement is taken as a reference for determinations of response. The nadir measurement is taken as a reference for PD; this measurement constitutes the smallest measurement recorded, including the baseline measurement if this is the smallest measurement.

Assessments regarding sites of disease in participants with lymphoma are based on clinical, laboratory and radiographic evaluation. Positron emission tomography (PET)-computed tomography (CT) scan is used for fluorodeoxyglucose (FDG)-avid lymphomas. For lymphomas that are not FDG-avid, anatomic contrast-enhanced CT scans is used. Bone marrow assessments are based on morphologic evaluation of bone marrow biopsies. Immunohistochemistry is used to assess response if the sample is indeterminate by morphology.

The anti-tumor activity of FT522 is evaluated based on assessed objective response rate (ORR) at any time on study. For participants with R/R BCL, disease response is assessed and classified into complete response (CR), partial response (PR), stable disease (SD), progressive disease (PD), or not evaluable (NE). The best overall response (BOR) is summarized for the efficacy-evaluable population. The secondary endpoint of ORR, defined as the proportion of participants who achieve a PR or better, is summarized descriptively along with the exact 95% CI based on the Clopper-Pearson's method by treatment regimen.

For all participants, secondary time-to-event endpoints include duration of response (DOR), duration of complete response (DoCR), PFS (Progression-free survival), and overall survival. Time-to-event endpoints are summarized using Kaplan-Meier methods. Kaplan-Meier plots are presented by treatment regimen and dose cohort. The number of events and the number of censored participants are summarized, and the median time-to-event and respective 95% CI are based on the Brookmeyer-Crowley method.

The PK (pharmacokinetic) analysis of FT522 is assessed by the detection of FT522 in peripheral blood following FT522 administration. Descriptive summaries including sample size (n), mean, standard deviation, standard error, median, Q1, Q3, and minimum and maximum values are provided for maximum observed concentration (Cₘₐₓ) and area under the curve (AUC).

Exploratory analyses include assessments of potential predictive and prognostic biomarkers in peripheral blood or serum and tumor tissue and bone marrow biopsies, as well as changes in the tumor microenvironment, as appropriate. The PK of FT522 in tumor biopsies is summarized using descriptive statistics. The association between baseline clinical and tumor characteristics, safety, and anti-tumor activity of FT522 are explored. The association of PK and pharmacodynamics of FT522 with safety and anti-tumor activity are also assessed. Lastly, the descriptive summaries of ctDNA measurements are additionally provided.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating a subject having lymphoma, comprising:
administering to the subject at least a first cycle of an adoptive cell therapy product, with the first cycle comprising one or more doses of the adoptive cell therapy product administered in a first effective amount at a preselected frequency, and with an option of administering one or more additional cycles with one or more doses in a second effective amount, during a course of treatment over a period of time;
wherein the first and the second effective amounts are the same or different;
wherein the adoptive cell therapy product comprises an engineered natural killer (NK) lineage cell comprising CD19-CAR (chimeric antigen receptor) expression, 41BB-ADR expression, exogenous CD16 expression, IL15RF expression, and CD38 knockout; and
wherein the lymphoma expresses CD19.

2. The method of claim 1, wherein the lymphoma
(i) is relapsed and/or refractory B-cell lymphoma (R/R BCL);
(ii) expresses CD20; and/or
(iii) is follicular lymphoma (FL), non-Hodgkin lymphoma (NHL), transformed indolent NHL, diffuse large B cell lymphoma (DLBCL), high-grade BCL (HGBCL), or primary mediastinal BCL.

3. The method of claim 1, wherein the subject has received at least one prior line of treatment comprising:
(i) an anti-CD20 monoclonal antibody therapy;
(ii) a CAR T-cell therapy; and/or
(iii) one of rituximab, obinutuzumab, chlorambucil, fludarabine, bendamustine, Hyper-CVAD, R-CHOP, or RDHAP, ibrutinib, acalabrutinib, zanubrutinib, pirtobrutinib, bortezomib venetoclax, lenalidomide, or combinations thereof.

4. The method of claim 1, wherein the course of treatment further comprises administering to the subject an effective amount of an anti-CD20 antibody.

5. The method of claim 4, wherein the course of treatment comprising the anti-CD20 antibody further comprises a conditioning chemotherapy.

6. The method of claim 4, wherein the anti-CD20 antibody comprises rituximab or a biosimilar thereof.

7. The method of claim 5, wherein the conditioning chemotherapy comprises:
(i) cyclophosphamide (CY);
(ii) fludarabine (FLU); or
(iii) bendamustine.

8. The method of claim 4, wherein the course of treatment comprising the anti-CD20 antibody does not comprise a conditioning chemotherapy.

9. The method of claim 4, wherein the anti-CD20 antibody is administered at a starting time prior to each of the first cycle and the one or more additional cycles of administering the adoptive cell therapy product.

10. The method of claim 5, wherein the conditioning chemotherapy is administered prior to day 1 of each cycle of administering the adoptive cell therapy product.

11. The method of claim 9, wherein the anti-CD20 antibody is rituximab or a biosimilar thereof, and is administered at a single dose of about 300 mg/m² to about 450 mg/m² to the subject about 2-6 days prior to day 1 of administering the adoptive cell therapy product in each cycle.

12. The method of claim 10, wherein the conditioning chemotherapy comprises cyclophosphamide and is administered at a daily dose of about 250 mg/m² to about 600 mg/m² and fludarabine at a daily dose of about 20 mg/m² to about 40 mg/m² for 3 consecutive days starting from about 4-6 days prior to day 1 of administering the adoptive cell therapy product in each cycle.

13. The method of claim 10, wherein the conditioning chemotherapy comprises bendamustine and is administered at a daily dose of about 30 mg/m² to about 100 mg/m² for 2 consecutive days starting from about 4-6 days prior to day 1 of administering the adoptive cell therapy product in each cycle.

14. The method of claim 1, wherein the engineered NK lineage cell is derived from an engineered induced pluripotent stem cell (iPSC) comprising a polynucleotide encoding a CD19-CAR, a polynucleotide encoding a 41BB-CAR, a polynucleotide encoding an exogenous CD16, a polynucleotide encoding IL15RF, and CD38 knockout.

15. The method of claim 1, wherein the first and second effective amounts of the adoptive cell therapy product in each dose is about 1×10⁸ cells to about 3×10⁹ cells, and is optionally escalated.

16. The method of claim 15, wherein the first and second effective amounts of the adoptive cell therapy product in each dose is about 1×10⁸ cells, about 3×10⁸ cells, about 9×10⁸ cells, about 2×10⁹ cells or about 3×10⁹ cells.

17. The method of claim 16, wherein if the effective amount in each dose results in a dose limiting toxicity (DLT) rate of 25% with an equivalence interval of 20%-30%, then the effective amount increases by 3 times or less to a higher dose.

18. The method of claim 1, wherein the method comprises administering the first and one or more additional cycles of the adoptive cell therapy over about 29 days with one dose each on day 1, 4, and 8.

19. The method of any one of claims 1-18, wherein the administering of the adoptive cell therapy product is (i) via intravenous infusion, and/or (ii) at a site of an outpatient setting; and/or wherein each dose of the adoptive cell therapy product is cryopreserved, and then thawed prior to administering; and wherein the adoptive cell therapy product is FT522.

20. A method of slowing progression of and/or treating BCL in a subject according to claims 1-19

21. The method of claim 20, wherein the subject has R/R BCL.

22. The method of claim 20, wherein the subject had no prior treatment of BCL.

23. The method of claim 20, comprising detecting and comparing one or more of the following at different given time points following administration of a first dose of the adoptive cell therapy:
(a) the presence of the engineered immune cell in a tumor of the subject;
(b) protein markers of disease in serum of the subject;
(c) cytokines in a peripheral blood sample from the subject;
(d) circulating tumor DNA in a peripheral blood sample from the subject; and
(e) lesion size and/or number,
wherein any of (a)-(e) is used to assess tumor burden, tumor immunobiology, and/or tumor therapy response, thereby determining efficacy of the multi-dose targeted adoptive cell therapy.


SEQUENCE LISTING (16 sequences)

SEQ ID NO: 1 - protein, 172 aa, synthetic construct
GLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 4 - protein, 455 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 494 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 455 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 7 - protein, 494 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 8 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDK

SEQ ID NO: 9 - protein, 379 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQ

SEQ ID NO: 10 - protein, 375 aa, synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYL

SEQ ID NO: 11 - protein, 575 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 18 aa, synthetic construct
MALPVTALLLPLALLLHA

SEQ ID NO: 13 - protein, 101 aa, synthetic construct
AELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYF

SEQ ID NO: 14 - protein, 90 aa, synthetic construct
KFMSTSVGDRVSVTCKASQNVGTNVAWYQQKPGQSPKPLIYSATYRNSGVPDRFTGSGSGTDFTLTITNVQSKDLADYFCQQYNRYPYTS

SEQ ID NO: 15 - protein, 263 aa, synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 308 aa, synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR